(19) United States
(12) Patent Application Publication
(10) Pub. No.: US 2007/0260270 A1
(43) Pub. Date: Nov. 8, 2007

(54) Cutter for preparing intervertebral disc space

(75) Inventors: Robert L. Assell, Wilmington, NC (US); Stephen D. Ainsworth, Wilmington, NC (US); Bradley J. Wessman, Wilmington, NC (US); Michael P. Barnhouse, Wilmington, NC (US); Brandon B. Arthurs, Wilmington, NC (US); Andrew H. Cragg, Edina, MN (US); Eugene A. Dickhudt, Lino Lakes, MN (US)

Correspondence Address: THE ECLIPSE GROUP, 10605 BALBOA BLVD., SUITE 300, GRANADA HILLS, CA, 91344, US

(73) Assignee: TranS1 Inc., Wilmington, NC (US)

(21) Appl. No.: 11/712,548

(22) Filed: Feb. 28, 2007

Related U.S. Application Data
(63) Continuation-in-part of application No. 11/586,338, filed on Oct. 24, 2006.
(63) Continuation-in-part of application No. 11/586,486, filed on Oct. 24, 2006.
(63) Continuation-in-part of application No. 10/972,184, filed on Oct. 22, 2004.
(63) Continuation-in-part of application No. 10/972,176, filed on Oct. 22, 2004.
(63) Continuation-in-part of application No. 10/972,077, filed on Oct. 22, 2004.
(63) Continuation-in-part of application No. 10/972,040, filed on Oct. 22, 2004.
(63) Continuation-in-part of application No. 10/972,039, filed on Oct. 22, 2004.
Application No. 11/586,338 is a continuation-in-part of application No. 11/259,614, filed on Oct. 25, 2005.
Application No. 11/586,338 is a continuation-in-part of application No. 11/256,810, filed on Oct. 24, 2005.
Application No. 11/256,810 is a continuation-in-part of application No. 11/199,541, filed on Aug. 8, 2005.
Application No. 10/972,077 is a continuation-in-part of application No. 10/309,416, filed on Dec. 3, 2002, now Pat. No. 6,921,403.
Application No. 10/309,416 is a continuation-in-part of application No. 10/125,771, filed on Apr. 18, 2002, now Pat. No. 6,899,716.
Application No. 10/125,771 is a continuation-in-part of application No. 09/848,556, filed on May 3, 2001, now Pat. No. 7,014,633.
Application No. 09/848,556 is a continuation-in-part of application No. 09/782,583, filed on Feb. 13, 2001, now Pat. No. 6,558,390.
(60) Provisional application No. 60/778,035, filed on Feb. 28, 2006.
(60) Provisional application No. 60/621,148, filed on Oct. 22, 2004.
(60) Provisional application No. 60/621,730, filed on Oct. 25, 2004.
(60) Provisional application No. 60/558,069, filed on Mar. 31, 2004.
(60) Provisional application No. 60/558,069, filed on Mar. 31, 2004.
(60) Provisional application No. 60/513,899, filed on Oct. 23, 2003.
(60) Provisional application No. 60/558,069, filed on Mar. 31, 2004.
(60) Provisional application No. 60/558,069, filed on Mar. 31, 2004.
(60) Provisional application No. 60/182,748, filed on Feb. 16, 2000.

(51) Int. Cl. A61B 17/32

(52) U.S. Cl. 606/171

(57) ABSTRACT

Cutter assemblies for use with cutter blades made of shape memory materials. The cutter blades may be deployed in the interior of an intervertebral disc space and rotated relative to a central axis of the cutter assembly to cut the material present for removal from the intervertebral disc space. Cutter blades with different attributes (such as throw length, cutter blade angle, type and location of blade edges) are adapted to achieve different objectives within the intervertebral disc space. Some cutter blades are adapted to promote bleeding of cartilage and vertebral body endplates and some cutter blades are adopted to avoid causing such bleeding. Closed loop cutter blades are described which have certain desirable attributes including the ability to remove the entire cutter blade from the intervertebral disc space after a break in the blade. Serration patterns are disclosed including a serration pattern that makes use of trapezoidal serrations.

This application builds upon a series of applications filed on behalf of assignee. In particular this application extends the innovative work in the area of manipulating material in the spine described in co-pending and commonly assigned U.S. patent application Ser. No. 10/972,077 for Method and Apparatus for Manipulating Material in the Spine filed Oct. 22, 2004 and subsequently published as United States Patent Application No. US 2005/0149034 A1 and U.S. Provisional Patent Application No. 60/778,035 for Method and Apparatus for Tissue Manipulation and Extraction filed Feb. 28, 2006. This application claims priority and incorporates in their entirety by reference both the '077 application and the '035 application. This application claims priority and incorporates by reference various applications claimed as priority documents by the '077 application specifically: U.S. Provisional Patent Application No. 60/513,899, filed on Oct. 23, 2003, and U.S. patent application Ser. No. 10/309,416, filed on Dec. 3, 2002 (now U.S. Pat. No. 6,921,403), which is a continuation-in-part of U.S. patent application Ser. No. 10/125,771, filed on Apr. 18, 2002 (now U.S. Pat. No. 6,899,716), which is a continuation-in-part of U.S. patent application Ser. No. 09/848,556, filed on May 3, 2001, (now U.S. Pat. No. 7,014,633) which is a continuation-in-part of U.S. patent application Ser. No. 09/782,583, filed on Feb. 13, 2001 (now U.S. Pat. No. 6,558,390), which claims priority to U.S. Provisional Patent Application No. 60/182,748, filed on Feb. 16, 2000. U.S. patent application Ser. No. 09/782,534 teaches various types of techniques for using cutting tools for removing disc material and preparation of spinal treatment sites that comprise a spinal disc, for example, a method of removing at least a portion of the nucleus through an anterior tract axial bore while leaving the annulus fibrosus intact.

This application extends the innovative work in the area of spinal motion preservation assemblies described in co-pending and commonly assigned U.S. patent application Ser. No. 11/586,338 for Spinal Motion Preservation Assemblies filed Oct. 24, 2006 and U.S. patent application Ser. No. 11/586,486 for Methods and Tools for Delivery of Spinal Motion Preservation Assemblies filed Oct. 24, 2006. This application claims priority to and incorporates by reference both '338 and the '486 application.

The '338 application claims priority to U.S. patent application Ser. No. 11/256,810 for Spinal Motion Preservation Assemblies and U.S. patent application Ser. No. 11/259,614 Driver Assembly for Simultaneous Axial Delivery of Spinal Implants. This application claims priority and incorporates by reference both the '810 application and the '614 application. This application claims priority and incorporates by reference two provisional applications claimed as priority documents by the '810 application specifically, U.S. Provisional Application No. 60/621,148 filed Oct. 22, 2004 for Spinal Mobility Preservation Assemblies and U.S. Provisional Application No. 60/621,730 filed Oct. 25, 2004 for Multi-Part Assembly for Introducing Axial Implants into the Spine. This application claims priority and incorporates by reference four co-pending and commonly assigned U.S. patent application Ser. Nos. 10/972,184, 10/972,039, 10/972,040, and 10/972,176 all filed on Oct. 22, 2004. These four applications claim priority to another U.S. Provisional Applications, Application No. 60/558,069 filed Mar. 31, 2004. Priority to this provisional is claimed through the four co-pending applications and the provisional is incorporated by reference. This application also claims priority through the '810 application to U.S. patent application Ser. No. 11/199,541 filed Aug. 8, 2005 and U.S. Provisional Application No. 60/599,989 filed Aug. 9, 2004 which is claimed as a priority document for the '541 application. Both of these applications are incorporated by reference.

While a number of applications have been incorporated by reference to provide additional detail it should be noted that these other applications (including those that have subsequently issued as patents) were written at an earlier time and had a different focus from the present application. Thus, to the extent that the teachings or use of terminology differ in any of these incorporated applications from the present application, the present application controls.

### BACKGROUND OF THE INVENTION

1. Field of the Invention

This invention relates generally to improved cutters and methods for preparing treatment sites within the spine, such at the intervertebral space between two adjacent vertebral bodies for subsequent therapeutic procedures including therapies where fusion of the two adjacent vertebral bodies is not desired such as therapies for the implantation of motion preservation devices into the spine.

2. Overview

The present invention is an extension of work in a series of patent applications (some now issued patents) with a common assignee. Much of the work is described in great detail in the many applications referenced above and incorporated by reference into this application. Accordingly, the background of the invention provided here does not repeat all of the detail provided in the earlier applications, but instead highlights how the present invention adds to this body of work.

The spinal column is a complex system of bone segments (vertebral bodies and other bone segments) which are in most cases separated from one another by discs in the intervertebral spaces (sacral vertebrae are an exception). FIG. 1 shows the various segments of a human spinal column as viewed from the side. In the context of the present disclosure, a “motion segment” includes adjacent vertebrae, i.e., an inferior and a superior vertebral body, and the intervertebral disc space separating said two vertebral bodies, whether denucleated space or with intact or damaged spinal discs. Unless previously fused (or damaged), each motion segment contributes to the overall flexibility of the spine contributes to the overall ability of the spine to flex to provide support for the movement of the trunk and head.

The vertebrae of the spinal cord are conventionally subdivided into several sections. Moving from the head to the tailbone, the sections are cervical 104, thoracic 108, lumbar 112, sacral 116, and coccygeal 120. The individual vertebral bodies within the sections are identified by number starting at the vertebral body closest to the head. The trans-sacral approach is well suited for access to vertebral bodies in the lumbar section and the sacral section. As the various vertebral bodies in the sacral section are usually fused together in adults, it is sufficient and perhaps more descriptive to merely refer to the sacrum rather than the individual sacral components.

It is useful to set forth some of the standard medical vocabulary before getting into a more detailed discussion of the background of the present invention. In the context of the this discussion: anterior refers to in front of the spinal column; (ventral) and posterior refers to behind the column (dorsal); cephalad means towards the patient's head (sometimes “superior”); caudal (sometimes “inferior”) refers to the direction or location that is closer to the feet. As the present application contemplates accessing the various vertebral bodies and intervertebral spaces through a preferred approach that comes in from the sacrum and moves towards the head, proximal and distal are defined in context of this channel of approach. Consequently, proximal is closer to the beginning of the channel and thus towards the feet or the surgeon, distal is further from the beginning of the channel and thus towards the head, or more distant from the surgeon. When referencing tools including cutters, distal would be the end intended for insertion into the access channel and proximal refers to the other end, generally the end closer to the handle for the tool.

The individual motion segments within the spinal columns allow movement within constrained limits and provide protection for the spinal cord. The discs are important to cushion and distribute the large forces that pass through the spinal column as a person walks, bends, lifts, or otherwise moves. Unfortunately, for a number of reasons referenced below, for some people, one or more discs in the spinal column will not operate as intended. The reasons for disc problems range from a congenital defect, disease, injury, or degeneration attributable to aging. Often when the discs are not operating properly, the gap between adjacent vertebral bodies is reduced and this causes additional problems including pain.

A range of therapies have been developed to alleviate the pain associated with disc problems. One class of solutions is to remove the failed disc and then fuse the two adjacent vertebral bodies together with a permanent but inflexible spacing, also referred to as static stabilization. One estimate is that in 2004 there were an estimated 300,000 fusion operations throughout the world. Fusing one section together ends the ability to flex in that motion segment. While the loss of the normal physiologic disc function for a motion segment through fusion of a motion segment may be better than continuing to suffer from the pain, it would be better to alleviate the pain and yet retain all or much of the normal performance of a healthy motion segment.

Another class of therapies attempts to repair the disc so that it resumes operation with the intended intervertebral spacing and mechanical properties. One type of repair is the replacement of the original damaged disc with a prosthetic disc. This type of therapy is called by different names such as dynamic stabilization or spinal motion preservation.

The Operation of the Spine

The bodies of successive lumbar, thoracic and cervical vertebrae articulate with one another and are separated by the intervertebral spinal discs. Each spinal disc includes a fibrous cartilage shell enclosing a central mass, the “nucleus pulposus” (or “nucleus” herein) that provides for cushioning and dampening of compressive forces to the spinal column. The shell enclosing the nucleus includes cartilaginous endplates adhered to the opposed cortical bone endplates of the cephalad and caudal vertebral bodies and the “annulus fibrosus” (or “annulus” herein) includes multiple layers of opposing collagen fibers running circumferentially around the nucleus pulposus and connecting the cartilaginous endplates. The natural, physiological nucleus includes hydrophilic (water attracting) mucopolysacharides and fibrous strands (protein polymers). The nucleus is relatively inelastic, but the annulus can bulge outward slightly to accommodate loads axially applied to the spinal motion segment.

The intervertebral discs are anterior to the spinal canal and located between the opposed end faces or endplates of a cephalad and a caudal vertebral bodies. The inferior articular processes articulate with the superior articular processes of the next succeeding vertebra in the caudal (i.e., toward the feet or inferior) direction. Several ligaments (supraspinous, interspinous, anterior and posterior longitudinal, and the ligamenta flava) hold the vertebrae in position yet permit a limited degree of movement. The assembly of two vertebral bodies, the interposed, intervertebral, spinal disc and the attached ligaments, muscles and facet joints is referred to as a “spinal motion segment”

The relatively large vertebral bodies located in the anterior portion of the spine and the intervertebral discs provide the majority of the weight bearing support of the vertebral column. Each vertebral body has relatively strong, cortical bone layer forming the exposed outside surface of the body, including the endplates, and weaker, cancellous bone in the center of the vertebral body.

The nucleus pulposus that forms the center portion of the intervertebral disc consists of 80% water that is absorbed by the proteoglycans in a healthy adult spine. With aging, the nucleus becomes less fluid and more viscous and sometimes even dehydrates and contracts (sometimes referred to as “isolated disc resorption”) causing severe pain in many instances. The spinal discs serve as “dampeners” between each vertebral body that minimize the impact of movement on the spinal column, and disc degeneration, marked by a decrease in water content within the nucleus, renders discs ineffective in transferring loads to the annulus layers. In addition, the annulus tends to thicken, desiccate, and become more rigid, lessening its ability to elastically deform under load and making it susceptible to fracturing or fissuring, and one form of degeneration of the disc thus occurs when the annulus fissures or is torn. The fissure may or may not be accompanied by extrusion of nucleus material into and beyond the annulus. The fissure itself may be the sole morphological change, above and beyond generalized degenerative changes in the connective tissue of the disc, and disc fissures can nevertheless be painful and debilitating. Biochemicals contained within the nucleus are enabled to escape through the fissure and irritate nearby structures.

Various other surgical treatments that attempt to preserve the intervertebral spinal disc and to simply relieve pain include a “discectomy”, or “disc decompression” to remove some or most of the interior nucleus thereby decompressing and decreasing outward pressure on the annulus. In less invasive microsurgical procedures known as “microlumbar discectomy” and “automated percutaneous lumbar discectomy”, the nucleus is removed by suction through a needle laterally extended through the annulus. Although these procedures are less invasive than open surgery, they nevertheless suffer the possibility of injury to the nerve root and dural sac, perineural scar formation, re-herniation of the site of the surgery, and instability due to excess bone removal. In addition, they generally involve the perforation of the annulus.

Although damaged discs and vertebral bodies can be identified with sophisticated diagnostic imaging, existing surgical interventions and clinical outcomes are not consistently satisfactory. Furthermore, patients undergoing such fusion surgery experience significant complications and uncomfortable, prolonged convalescence. Surgical complications include disc space infection; nerve root injury; hematoma formation; instability of adjacent vertebrae, and disruption of muscle, tendons, and ligaments, for example.

Several companies are pursuing the development of prosthesis for the human spine, intended to completely replace a physiological disc, i.e., an artificial disc. In individuals where the degree of degeneration has not progressed to destruction of the annulus, rather than a total artificial disc replacement, a preferred treatment option may be to replace or augment the nucleus pulposus, involving the deployment of a prosthetic disc nucleus. As noted previously, the normal nucleus is contained within the space bounded by the bony vertebrae above and below it and the annulus fibrosus, which circumferentially surrounds it. In this way the nucleus is completely encapsulated and sealed with the only communication to the body being a fluid exchange that takes place through the bone interface with the vertebrae, known as the endplates.

The hydroscopic material found in the physiological nucleus has an affinity for water (and swells in volume) which is sufficiently powerful to distract (i.e., elevate or “inflate”) the intervertebral disc space, despite the significant physiological loads that are carried across the disc in normal activities. These forces, which range from about 0.4× to about 1.8× body weight, generate local pressure well above normal blood pressure, and the nucleus and inner annulus tissue are, in fact, effectively avascular.

Details of specific advantages and specific motion preservation devices including methods for implanting motion preservation devices are described in various pending applications including Ser. Nos. 11/586,338 and 11/586,486 referenced above. The reader may select to read these details but there is not a need to repeat that material in its entirety here.

While the cutters described below may be used in other surgical procedures including spinal surgery that does not approach an intervertebral space via an axial approach but comes to the space through an anterior or a posterior approach. The cutters may be used in surgical procedures with the motion preservation devices inserted axially within the spine, following either partial or complete nucleectomy and possibly through a cannula that is docked against the sacrum, into a surgically de-nucleated disc space, from said access point across a treatment zone. In such a procedure, the introduction of the spinal motion preservation assembly of the present disclosure is accomplished without the need to surgically create or deleteriously enlarge an existing hole in the annulus fibrosus of the disc.

Design of cutter blades includes considerations in many cases of the efficiency with which the cutter blade prepares the contents of the nucleus for removal by cutting (slicing, tearing, or some combination of the two). It is generally desirable to allow a surgeon to work quickly and efficiently to reduce the time of surgery which has benefits in reducing the use of expensive resources such as the surgical team and the surgical suite and also reduces the length of time that a patient is kept under anesthesia.

A cutter blade that must be replaced frequently may be less desirable than a cutter blade with similar characteristics that is more durable and thus may be used longer without needing to be replaced.

A cutter blade that fails in a mode where all the pieces of the failed cutter blade may be easily removed from the intervertebral disc space and the patient body may be preferred over a similar cutter blade that does not have this characteristic.

### SUMMARY OF THE DISCLOSURE

Disclosed herein are cutter assemblies for use with cutter blades made of shape memory materials. The cutter blades may be deployed in the interior of an intervertebral disc space and rotated relative to a central axis of the cutter assembly which is substantially aligned with a centerline of an axial channel. Rotation of a cutter blade as part of a cutter assembly within an intervertebral disc space cuts the material present there for removal from the intervertebral disc space. Cutter blades with different attributes (such as throw length, cutter blade angle, type and location of blade edges) are adapted to achieve different objectives within the intervertebral disc space. Some cutter blades are adapted to promote bleeding of cartilage and vertebral body endplates and some cutter blades are adopted to avoid causing such bleeding as different therapeutic procedures seek or seek to avoid such bleeding.

Closed loop cutter blades are described which have certain desirable attributes including the ability to remove the entire cutter blade from the intervertebral disc space after a break in the blade. Serration patterns are disclosed including a serration pattern that makes use of trapezoidal serrations.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The invention can be better understood with reference to the following figures. The components in the figures are not necessarily to scale, emphasis instead being placed upon illustrating the principles of the invention. Moreover, in the figures, like reference numerals designate corresponding parts throughout the different views.

FIG. 1 identifies the sections of a human spine.

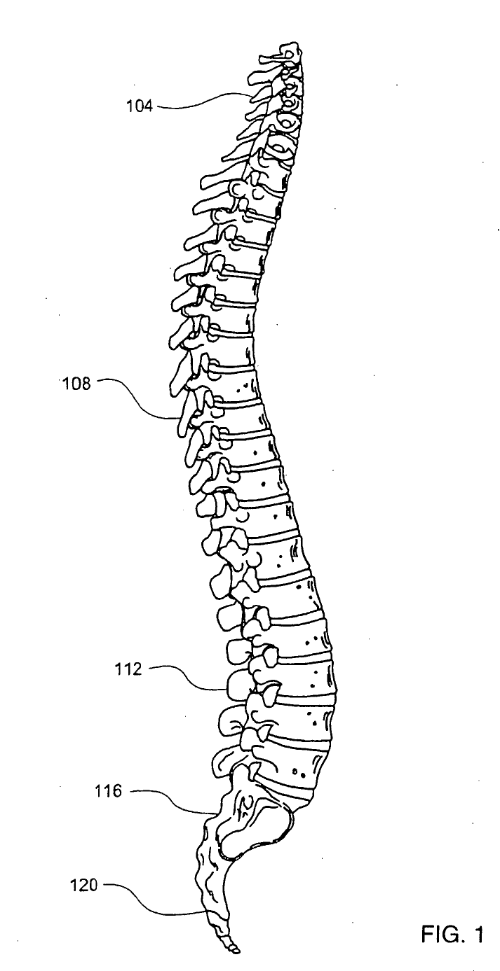

FIGS. 2(A)-(C) illustrates an anterior trans-sacral axial access method of creating an axial channel in the spine which can be used to prepare an axial channel in the spine for use with the present disclosure.

FIG. 3 shows a cutter assembly inserted into an axial channel with the cutter blade in an extended position.

FIGS. 4A-4B are views of a cutter assembly.

FIG. 5A-5B shows one method for connecting a cutter blade to a cutter shaft.

FIG. 6A-6D provides additional views of a cutter assembly including stops that limit the range of travel of the cutter sheath.

FIG. 7 addresses the concept of a series of cutter blades of different throw lengths within an intervertebral disc space.

FIG. 8 introduces the issues that arise when the axial channel is not substantially perpendicular to the endplates for an intervertebral disc space.

FIG. 9 shows blade arms for cutter blades with a angles of 45 degrees, 90 degrees, and 135 degrees with respect to a longitudinal portion of the cutter blade.

FIGS. 10A-10C show three views a cutter blade.

FIG. 12 shows a cutter blade in a first cutter shaft without cutter shaft extensions.

FIG. 13 shows a cutter blade in a cutter shaft with cutter shaft extensions.

FIGS. 14-16 show views of a cutter shaft without a cutter shaft extensions.

FIGS. 17-19 show views of a cutter shaft with cutter shaft extensions.

FIGS. 20A-20C are views of a cutter blade with round serrations on one side of the cutter blade.

FIGS. 21A-21C are views of a cutter blade with round serrations on one side of the cutter blade and a different serration pattern at the cutter blade tip.

FIGS. 22A-22C show several views of a cutter blade with serrated cutting edges on both the clockwise side of the cutter blade and counterclockwise side of the cutter blade.

FIG. 23 and FIG. 24 show details for adding the serrations to flat stock of appropriate size for creation of a cutter blade.

FIG. 25 notes the material to be removed from the flat stock to create a cutter blade using a trapezoid serration pattern.

FIG. 26 shows a cutter blade with a trapezoid serration pattern with the cutter blade in a cutter assembly.

FIGS. 27A-27D show views of blade stock with a trapezoidal serration pattern.

FIGS. 28A-D shows a rounded tooth serration pattern cut into blade stock.

FIGS. 29A-G show a variety of views of a cutter blade made from blade stock with a serrations pattern like that shown in FIGS. 28A-D.

FIGS. 30 and 31 show two examples of cutter blades with a 135 degree angle between the proximal portion of the blade arm and the longitudinal portion of the cutter blade.

FIG. 32A-32C shows a rivet based connection of a cutter blade to a cutter shaft.

### DETAILED DESCRIPTION

While the inventive cutters described below may be used in other surgical procedures, it is useful in context to describe how these cutters could be adopted for use in a trans-sacral approach. As noted above there are many advantages associated with a minimally invasive, low trauma trans-sacral axial approach. The trans-sacral axial approach (described and disclosed in commonly assigned U.S. Pat. Nos. 6,558,386; 6,558,390; 6,575,979; 6,921,403; 7,014,633, and 7,087,058) has a number of advantages over other routes for delivery of therapeutic devices to motion segments but there are logistical challenges to the preparation of an intervertebral disc space via an axial access channel. The process of addressing these challenges impacts certain aspects of the cutters intended for use in this manner.

Trans-Sacral Axial Access

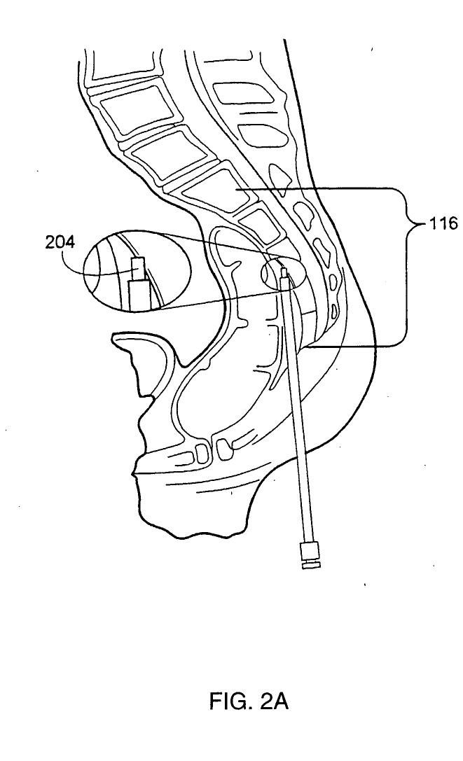

The trans-sacral axial access method illustrated in FIG. 2, eliminates the need for muscular dissection and other invasive steps associated with traditional spinal surgery while allowing for the design and deployment of new and improved instruments and therapeutic interventions, including stabilization, motion preservation, and fixation devices/fusion systems across a progression-of-treatment in intervention.

FIG. 2 provides an introductory overview of the process with FIGS. 2(a) and 2(b) showing the process of “walking” a blunt tip stylet 204 up the anterior face of the sacrum 116 to the desired position on the sacrum 116 while monitored one or more fluoroscopes (not shown). This process moves the rectum 208 out of the way so that a straight path is established for the subsequent steps. FIG. 2(c) illustrates a representative trans-sacral axial channel 212 established through the sacrum 116, the L5/sacrum intervertebral space, and into the L5 vertebra 216. If therapy is being provided to the L4/L5 motion segment then the channel would continue through the L5 vertebra 216 through the L4/L5 intervertebral space, and into the L4 vertebra 220.

The discussion of FIG. 2 is provided to provide context for the present disclosure. Previous applications (some now issued as U.S. patents) with common assignee have included a description of an alternative access method that is a posterior trans-sacral axial spinal approach rather than an anterior trans-sacral axial spinal approach. (See e.g. U.S. Pat. No. 6,558,386 for Axial Spinal Implant and Method and Apparatus for Implanting an Axial Spinal Implant Within the Vertebrae of the Spine as this patent describes the anterior trans-sacral axial approach illustrated in FIG. 2 and is incorporated by reference in its entirety.)

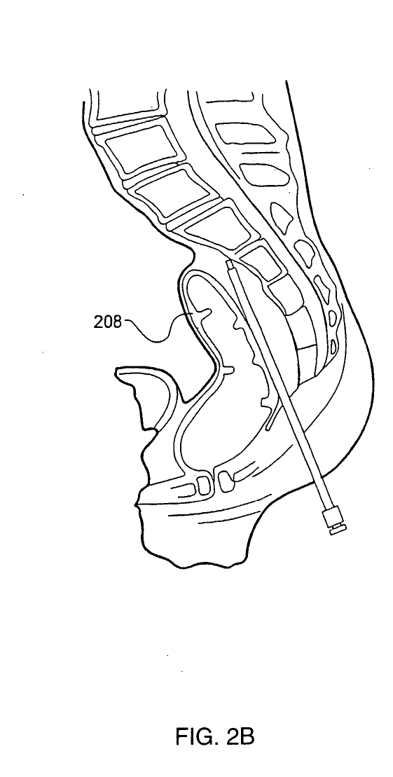

Referring to FIG. 3, a cutter 400 is inserted through the axially aligned anterior tract 372 defined by the lumen of the dilator sheath 380 and the axial channel 212 which is difficult to see as the dilator sheath 380 substantially fills the axial channel 212 as it passes through the sacrum 116. (One of skill in the art will appreciated that the axial channel 212 may be extended axially by a sequence of steps so that the length of an axial channel in one Figure may be different from another Figure such that the axial tract may include additional vertebral bodies or intervertebral disc spaces). One of skill in the art will appreciate that due to anatomical differences the axial channel for some therapies may miss the sacrum and may enter through another portion of the spine.

As shown in FIG. 3, motion segment 316 that includes the proximal vertebra 308 (the sacrum 116), the intervertebral space 312 (in this case the L5-S1 space with disc 330, annulus fibrosus 334 and nucleus 338), the distal vertebra 304 (in this case L5 216). The cutter 400 comprises a cutting blade (e.g., cutter blade 453 which refers collectively to any blade configuration) which is remotely manipulable. The manipulations of the cutter blade 453 may include retracting the cutter blade 453 into the cutter assembly 400 so that the maximum radius of the cutter assembly 400 is reduced and the cutter assembly with the retracted blade 453 may be advanced through the axial channel 212. After reaching the location where the cutter blade 453 is to be operated, the cutter blade 453 may be extended.

As shown in FIG. 3, the centerline 262 of the cutter 400 is very close to the centerline of the axial channel 212 due to the fit of the dilator sheath 380 in the axial channel 212 and the fit of the cutter 400 within the dilator sheath 380. When the cutter blade 453 is extended as shown in FIG. 3 the cutter blade is substantially transverse to the centerline 262 of the cutter 400. The extended cutter blade 453 is extended laterally into the nucleus 338 of the spinal disc 330.

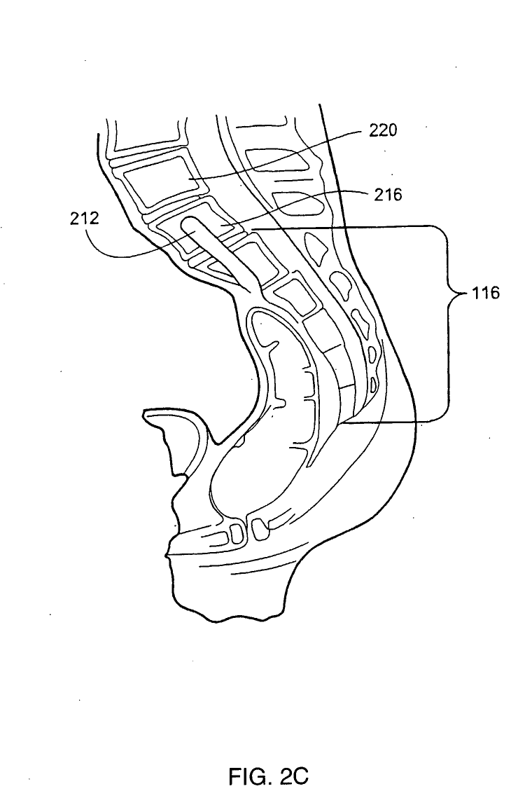

The cutter shaft 410, cutter sheath 430 (shown in FIG. 4) and the handle components are preferably co-configured to enable the cutter blade 453 and the cutter shaft 410 to which it is attached be able to be “pushed-pulled” so as to retract the cutter blade 453 into the cutter sheath and then extend the cutter blade 453 from the distal end of the cutter sheath as needed. More specifically, the cutter blade edges(s) of the cutter blade 453 are retracted into the cutter sheath 430 (FIG. 4) for delivery into the intervertebral disc space 312. Once the cutter 400 is in position, the cutter blade 453 is extended distally and rotated using the handle to cut tissue within the intervertebral disc space 312. After completing the cutting task or until the cutter blade needs replacement, the cutter blade 453 is again retracted into the cutter sheath 430 (FIG. 4) for removal of the cutter assembly unit 400 from the axial channel 212.

The cutter assembly 400, cutter blade 453 and cutter assembly shaft 410 are shown schematically in FIGS. 4A-4B and not necessarily to scale to one another or to the axial channel 212.

Cutters can be used to perform nucleectomies via insertion into a disc space to excise, fragment and otherwise loosen nucleus pulposus and cartilage from endplates from within the disc cavity and from inferior and superior bone endplate surfaces. As noted within this disclosure, damage to or removal of cartilage tends to cause bleeding within the intervertebral disc space 312. Bleeding tends to promote bone growth, which may be desired in a fusion type therapy but may be undesirable in other therapies, including therapies that call for the implantation of a motion preservation device into the motion segment 316.

With reference to the exemplary embodiments of FIGS. 4A-B, the cutter assembly 400 (also referred to as simply a cutter) includes: a cutter shaft 410 with a distal end 412 and a proximal end 414; a cutter blade 453 connected to the distal end 412 of the cutter shaft 410; a handle 416 connected to the proximal end 414 of the cutter shaft by an attachment process such as a set screw or pin; a cutter sheath 430 placed concentrically over the shaft 410; and a shaft sleeve 418 (shown in subsequent drawings).

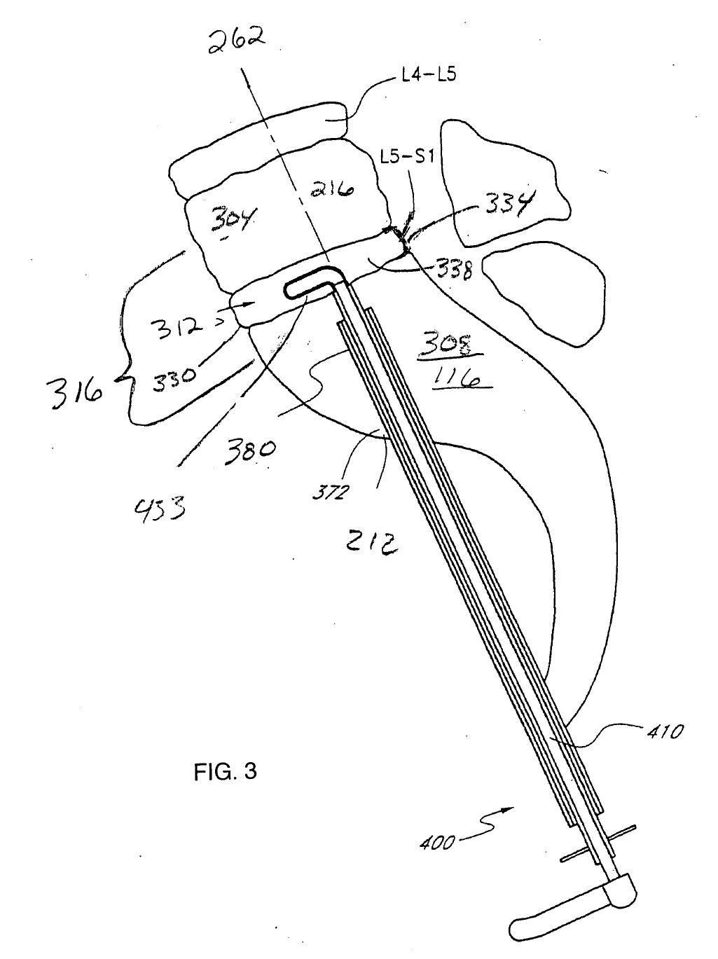

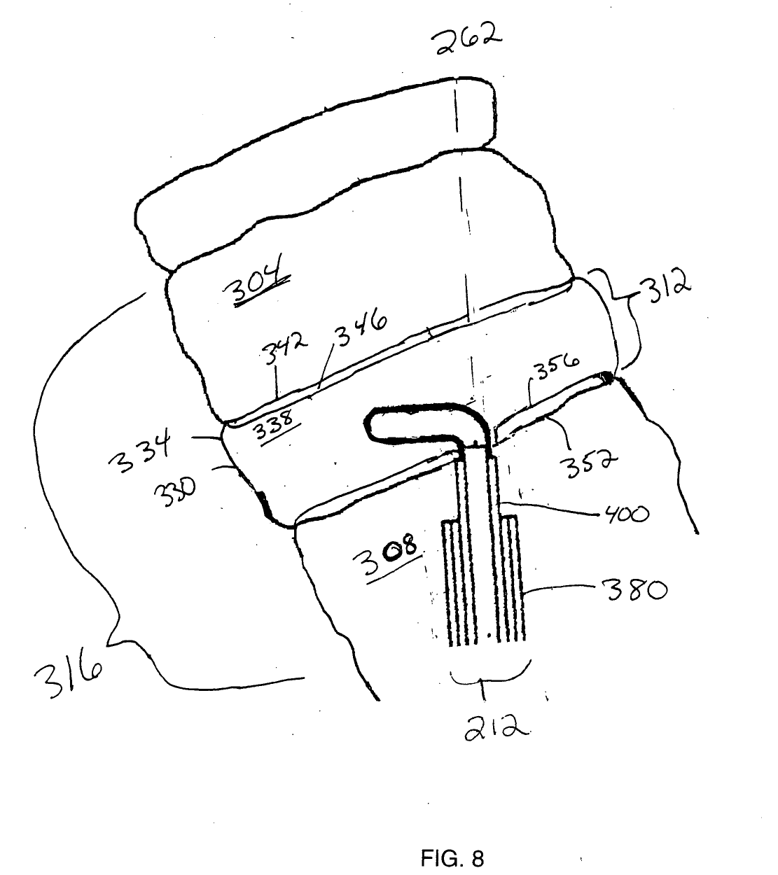

FIGS. 5A-5B illustrate one method of connecting a cutter blade 453 to a cutter shaft 410. Before the pin 409 is inserted, the longitudinal portion 406 of the cutter blade 453 is placed into a slot 413 near the distal end 412 of the cutter shaft 410. The cutter blade slot 427 may be aligned with the cutter shaft hole 411 within the shaft slot 413. A pin 409 may be placed through a shaft sleeve hole 419 in a shaft sleeve 418 and through a cutter blade slot 427 (visible in FIG. 5A), a cutter blade hole 407 on the opposite side of the longitudinal portion 406 of the cutter blade 453 (best seen in FIG. 10A). The pin passes through cutter blade hole 407 and into a cutter shaft hole 411 in a cutter shaft slot 413.

The shaft slot 413 is dimensioned to accommodate a cutter blade 453. The width of the slot 413 is approximately the same as the width of the longitudinal portion 406 of the cutter blade 453. The curvature 428 at the distal end of the slot 413 accommodates the curvature of the cutter blade 453 between the longitudinal portion 406 and the portion of the cutter blade that may be extended 402 (also known as the cutter blade arm 402) (which defines the reach or throw of the cutter blade 453). The slot 413 provides torsional support to the cutter blade arm 402 while the curvature 428 at the distal end of the slot 413 provides axial support to the cutter blade arm 402 to work in conjunction with cutter blade edge geometries to reinforce the cutter blade 453. The cutter shaft extension 480 discussed in more detail below provides additional support to the cutter blade 453 to reduce the tendency of the cutter blade to flex when rotated into tissue.

The shaft sleeve 418 when pinned, effectively serves to align and fix the shaft 410 and the longitudinal portion 406 of the cutter blade 453. For purposes of illustration, the pin 409 that fixes the cutter blade 453 to the shaft 410 may be approximately 0.06 inches (1.5 mm) in diameter.

As cutter blade hole 407 is pinned to the cutter blade shaft 410, the cutter blade 453 is affixed to the cutter blade shaft 410. The cutter blade slot 427 allows some relative motion of the slotted portion of the longitudinal portion 406 relative to the pinned portion of the longitudinal portion 406 to accommodate the change of shape of the cutter blade 453 as it goes from sheathed to extended and back to sheathed.

The rest of the cutter 400 components can be fixedly secured to each other using any known suitable fixation mechanisms.

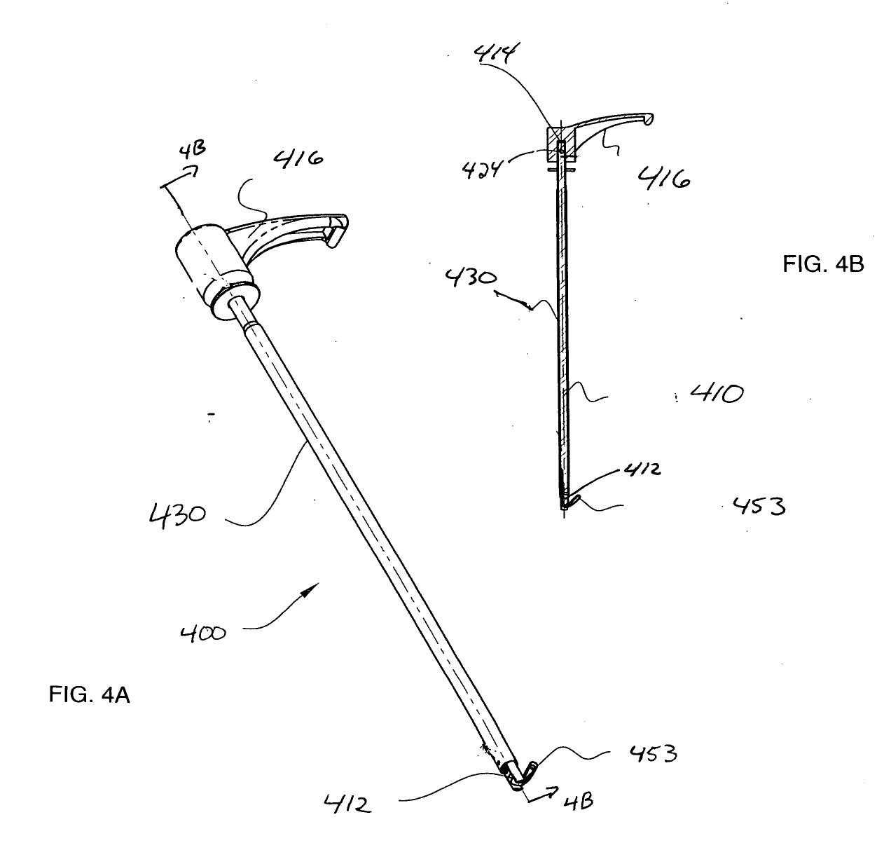

FIGS. 6A-6D provides a series of views of a cutter assembly 400. FIG. 6A is a top view of the cutter assembly 400. FIG. 6B is a rear view of the cutter assembly 400. FIG. 6C is a cross section of FIG. 6B. FIG. 6D is a enlarged portion of FIG. 6C.

As shown in FIGS. 6A and 6D, the slot in the cutter shaft 410 may be oriented so that the handle 416 is aligned with the blade arm 402 (when extended). While not required, this relationship between the handle and blade is a useful way to allow the surgeon to keep track of the position of the extended blade arm 402 by knowing rotational position of the handle 416.

As best seen in FIG. 6D, the travel range 440 of the cutter sheath 430 is limited at the proximal end by a proximal end stop 444 attached to the cutter shaft 410. The travel range 440 of the cutter sheath 430 is limited at the distal end by a shoulder 448 on the cutter shaft 410.

One of skill in the art will appreciate that while the cutter blades 453 are to be used with a single patient and then disposed, that, certain components such as the handle 416, cutter shaft 410, and cutter sheath 430 may be reusable. The handle and cutter shaft could be made as one integral component.

A sleeve or internal sheath liner (not shown) may be inserted inside the cutter sheath to reduce friction. The cutter blade 453 may be formed from a shape memory alloy including a nickel-titanium shape memory alloy such as Nitinol™. The cutter sheath 430 may be made from an appropriate grade of stainless steel. To reduce the friction between the cutter blade 453 and the inner surface of the cutter sheath 430, a dry lubrication such as poly-tetrafluoroethylene (PTFE) may be used. Alternatively, the sleeve or internal sheath liner may be made of a material with a coefficient of friction that is lower than the cutter blade. If this component is to be reused, it may be chosen for its ability to withstand multiple sterilization cycles. Ultra-high molecular weight polyethylene (UHMWPE) is one such material.

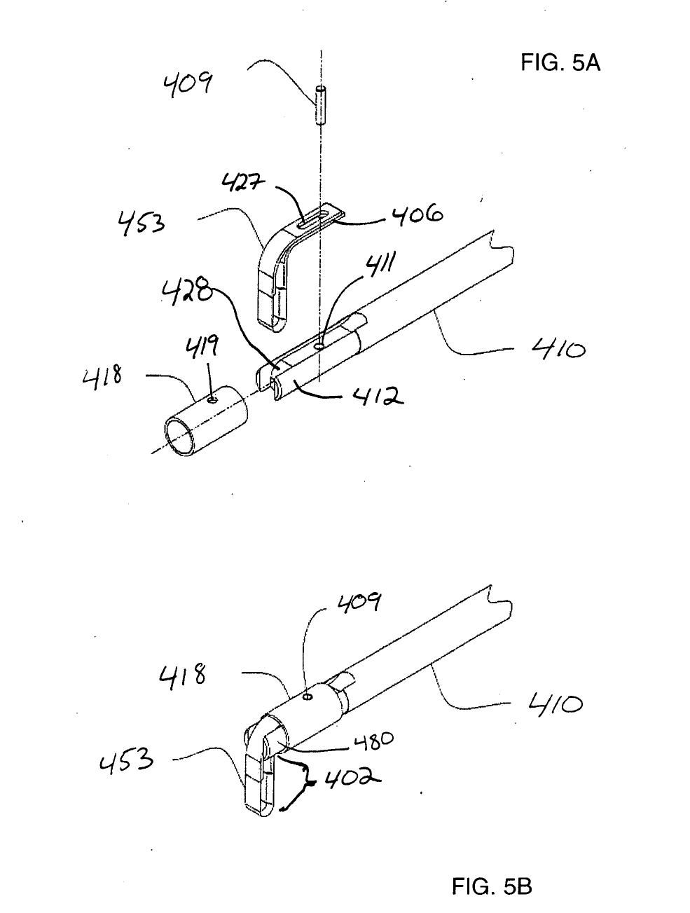

After this introduction to cutters and cutter components, it is useful to discuss why a sequence of cutters may be used while preparing the interior of an intervertebral disc space 312. FIG. 7 shows a first example. In FIG. 7 a motion segment 316 including a distal vertebral body 304, an intervertebral disc space 312 (with a intervertebral disc 330 including an annulus fibrosus 334, and nucleus pulposus 338 and bounded by the endplates), and a proximal vertebral body 308 are shown. For purposes of this example, it is not important which vertebral bodies are involved beyond the need for them to be adjacent vertebral bodies.

FIG. 7 includes the endplate 342 of the distal vertebral body 304 and a representation of the layer of cartilage 346 located on the endplate 342 which defines one portion of the intervertebral disc space 312. Assuming the route of access is a trans-sacral axial access, from the point of reference of the intervertebral disc space 312, endplate 342 would be the superior endplate. Likewise FIG. 7 includes the endplate 352 of the proximal vertebral body 308 and a representation of the layer of cartilage 356 located on the endplate 352 which defines one portion of the intervertebral disc space 312. Assuming the route of access is a trans-sacral axial access, from the point of reference of the intervertebral disc space 312, endplate 352 would be the inferior endplate.

One of skill in the art will recognize that the inclusion of the cartilage layers 346 and 356 is for purposes of discussing the use of cutters and is not intended to be an anatomically correct and appropriately dimensioned representation of cartilage.

The position of the cutter within the intervertebral disc space may be visible to the surgeon under real-time fluoroscopic imaging (possibly both anterior/posterior and lateral imaging).

In order to illustrate a point, FIG. 7 includes representations of three different cutter blades 504, 508, and 512 of differing throw lengths. One of ordinary skill in the art will appreciate that one method for cutting the nucleus 338 would use a series of cutter blades (504, 508, 512, and possibly another longer blade) to gradually cut the nucleus 338. One of ordinary skill in the art will understand that these three blades of different throw lengths (sometime called reaches) would be used sequentially from shorter to longer and it is only for the point of illustration that three different blade lengths are shown simultaneously in FIG. 7. To provide context, the reach of a series of cutter blades used in a particular procedure may range from 0.40 inches for a small cutter blade to 0.70 inches for a large cutter blade. One of skill in the art will recognize that these ranges are illustrative and could be different. It will be understood that the optimum throw for cutter blades depends on several factors, including patient anatomy and (axial) entrance point into the disc space, as well as issues related to sagittal symmetry of the spinal disc. Moreover, for safety reasons, it may be desirable to limit the length of the cutter blade to preclude a throw that is too close to the disc edge, in other words to avoid making contact between the cutter blade and the annulus fibrosus to preclude compromising the annulus fibrosus.

Note that the cutter blades 504, 508, and 512 when extended are transverse to the centerline of the cutter 262 and parallel to the axis 266 that is perpendicular to cutter blade centerline 262. The cutter blades are also close to parallel to the endplates 342 and 352 and the layers of cartilage 346 and 356.

In this example, the successively longer cutter blades 504 508, and 512, could be rotated 360 degrees or more around the centerline 262. Some surgeons may prefer to work on one segment at a time by rotating the cutter handle a fraction of 360 degrees (perhaps approximately 90 degrees) then rotating the cutter handle in the opposite direction to return to the position occupied by the cutter. Thus, the process tends to proceed while working on radial quadrants. Sometimes this short movement is compared to the movement of windshield wipers on an automobile.

In addition to using a series of cutter blades with sequentially increasing throws, the surgeon will need to adjust the axial position of the cutter blade by sliding the cutter forward (in the direction towards distal) relative to the motion segment so that the cutter blade move sequentially closer to the cartilage 346 on the endplate 342 on the distal vertebral body 304. The surgeon may opt to create a first space relatively close to the proximal vertebral body by using a sequence of cutters of increasing throws then repeating the process with the cutter extended further into the nucleus (and repeating the sequence of blades of increasing throws).

Alternatively, the surgeon may choose to use one or more cutters with a first throw to create a space approximating a cylinder that is substantially the height of the space between the two layers of cartilage and a radius approximately equal to a first blade throw. This process may involve the use of a radial cutter blade with a given throw length followed by one or more cutter blades at a different blade angle(s) (for example 45 degrees) but the same throw length. Once the cutting is complete for a given throw length, the surgeon moves to cutter blades of a longer throw length starting again with a radial cutter blade. This process may be repeated with cutter blades of increasing blade throws until the desired amount of space is created.

The nature of the therapeutic procedure and the patient anatomy will determine the maximum cutter blade throw length required. Certain procedures may tend to use a greater number of cutter blade throw lengths to make smaller incremental increases in throw length. Other procedures may simply use a small throw length then move to the maximum throw length needed to prepare the intervertebral disc space.

As the nucleus material is cut, the surgeon may periodically remove the cutter from the axial channel and use any appropriate tissue extractor tool. U.S. patent application Ser. No. 10/972,077 (referenced above) describes several retractable tissue extractors that may be used for this purpose.

U.S. patent application Ser. No. 10/972,077 (referenced above) noted that when preparing a intervertebral disc space for a fusion procedure, it can be advantageous to use cutters to scrape away the cartilaginous endplate and roughen the vascularized vertebral body so as to cause bleeding, which is desirable in order to facilitate bone growth and to promote fusion of the vertebral bodies of the relevant motion segment.

However, not all therapeutic procedures seek to obtain such bleeding to promote fusion. It is unavoidable to disturb the a portion of endplate 352 of the proximal vertebral body as the axial channel is created through the endplate 352 and it is likewise unavoidable to disturb a portion of the cartilage 356 in the immediate vicinity of the axial channel (likewise the endplate 342 and cartilage 346 of the distal vertebral body 304 if the axial channel 212 (FIG. 2C) is extended into the distal vertebral body 304). However, the unavoidable disturbance of a small portion of an endplate and cartilage does not remove the advantage within certain procedures of avoiding damage to other portions of the cartilage and endplate.

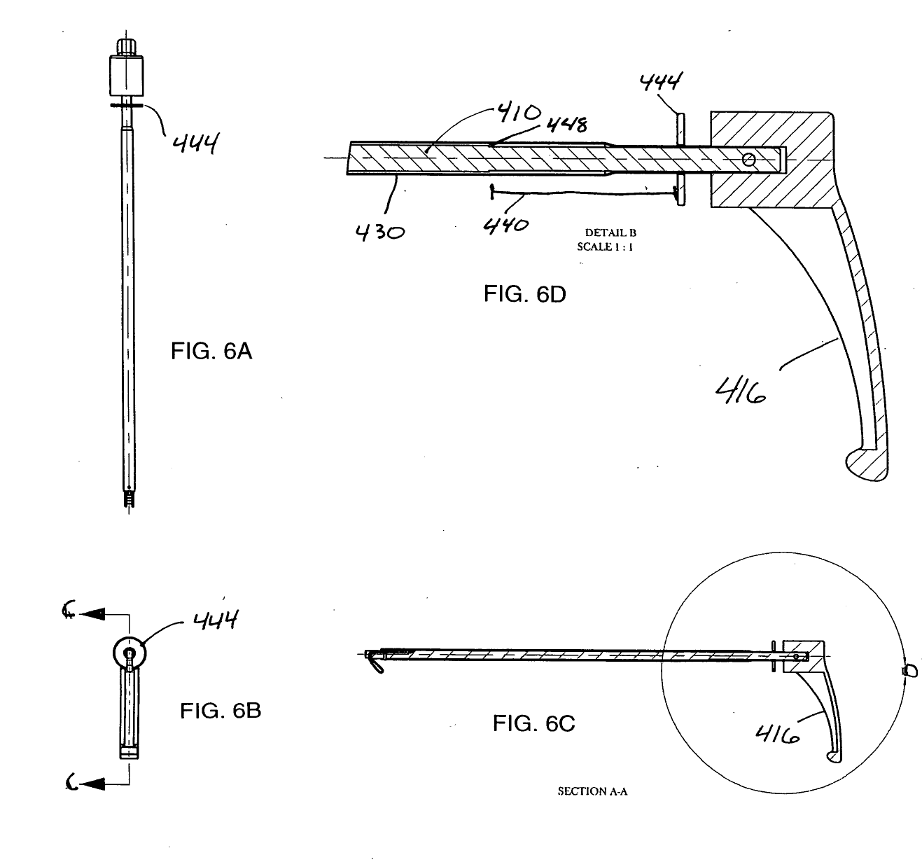

FIG. 8 depicts a different alignment between the axial channel 212 and the endplates of the two vertebral bodies. In FIG. 8, a cutter assembly 400 passed into and partially through a dilator sheath 380 in the axial channel 212 would have the cutter centerline 262 at an angle that is not close to perpendicular to the endplate 352 of the proximal vertebral body 308 or the endplate of the 342 of the distal vertebral body (the inferior and superior endplates of the intervertebral disc space 312).

A cutter blade 353 with an angle between the cutter shaft 310 and the cutter blade 353 of approximately 90 degrees would be useful in cutting a portion of the nucleus, but could not remove other portions of the nucleus. Cutter blades with an angle of 90 degrees are sometimes referenced as radial cutters.

FIG. 8 is intended to highlight the need for cutter blades with blade angles other than 90 degrees. FIG. 8 is not intended as an indication of an optimal alignment of an axial channel for any particular therapeutic procedure. In actual medical procedures, while planning the placement of a axial channel, the surgeon will evaluate and select an alignment that provides for appropriate clearance from anatomic structures to allow for safe and effective implantation including effective anchoring within the relevant vertebral bodies.

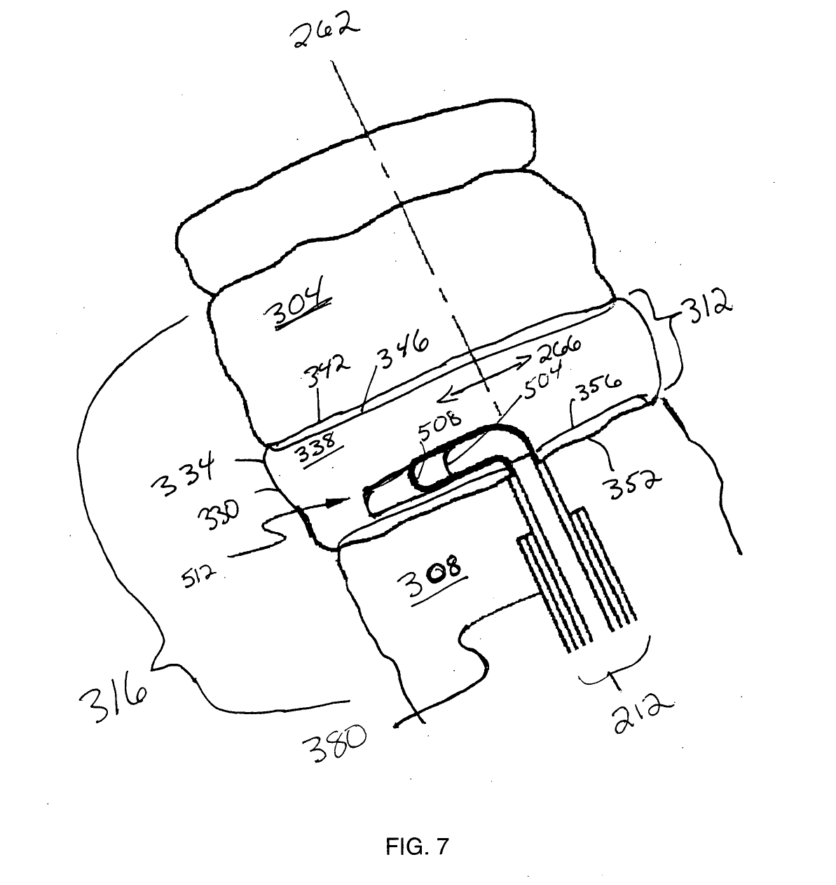

FIG. 9 illustrates a naming convention that is useful when discussing another attribute of cutter blades. In this case cutter blade 460 is a 90 degree cutter blade as there is a 90 degree angle (nominal) between the proximal side portion of the blade arm and the longitudinal portion 406 of the cutter blade 460. A portion of a 45 degree cutter blade 464 is shown with the more proximal portion of the portion of the cutter blade 464 at approximately 45 degrees with respect to the back of the longitudinal portion 406. While not shown here, an intermediate portion would connect the portion of the cutter blade 464 to a longitudinal portion 406.

Likewise a portion of a 135 degree cutter blade 468 is shown with the more proximal portion of the portion of the 135 degree cutter blade 468 at approximately 135 degrees with respect to the back of the longitudinal portion 406.

Note that as can be observed based on FIGS. 5 and 6, the longitudinal portion 406 of a cutter blade is going to be substantially parallel to the length of the cutter shaft 410 and the cutter sheath 430, and the centerline axis of the cutter 262 so that these lines could be used for measuring the cutter blade angle.

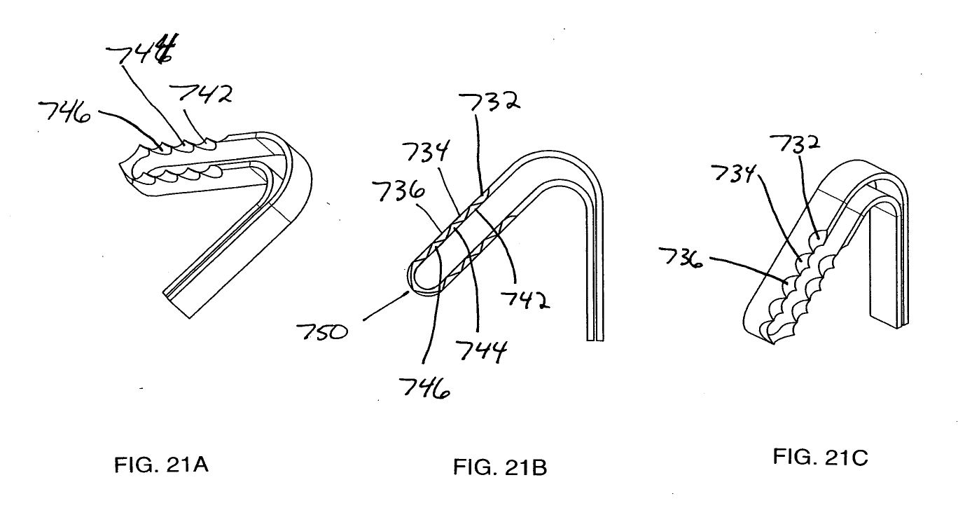

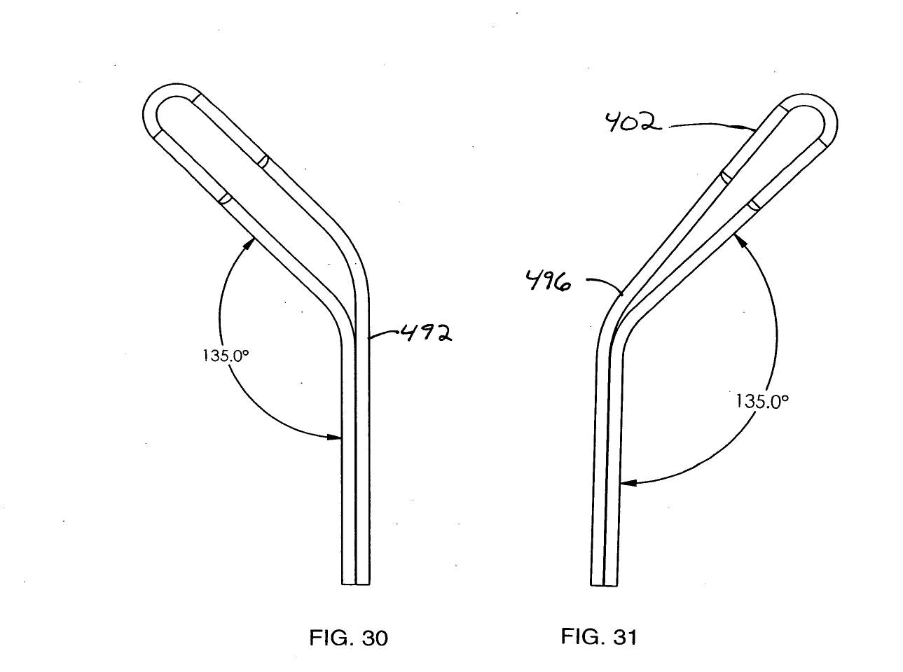

A complete 45 degree cutter blade is shown in FIG. 21. A complete 135 degree cutter blade is shown in FIGS. 30 and 31.

In some cutter blades, the proximal portion of the cutter blade does not run parallel with any angle reference line. In such case, it may be useful to simply measure the cutter blade angle based on the most proximal portion of the extended blade arm.

One of skill in the art will recognize that to the extent that the cutter blades are produced in a finite number of nominal cutter blade angles, the actual measurement of the precise angle may deviate a few degrees (perhaps 5) from the nominal angle value. The actual angle may deviate over cycles of moving from the sheathed to the extended position.

In many situations a set of cutter blades of various combinations of throw lengths and angles (such as 45 degree, 90 degree, and 135 degree) may be sufficient. Some surgeons may feel that they obtain adequate results for some therapies with using just 90 degree and 45 degree cutter blades. Other angles could be used, including angles that deviate less from 90 such as 60 and 120 degrees, or angles that deviate more from 90 degrees such as 25 and 155 degrees. Angles even closer to 90 degrees may be useful in some applications such as an angle in the vicinity of 105 degrees. Kits could include more than three angle values for the cutter blades. For example, a kit might include blades at 25, 45, 60, 90, 105, 120, 135 and 155 degree angles. With this range of blade angles, there is a wide variation of the extent to which the extended blades are transverse to the long axis of the cutter assembly, but in all these cases the cutter blades are significantly transverse to the long axis of the cutter assembly and to the longitudinal portions of the cutter blades.

Some surgeons may work on a situation such as presented in FIG. 8 by initially using a short 90 degree cutter blade, then using progressively longer 90 degree cutter blades (one or more longer cutter blades) to cut as much material within the intervertebral disc space 312 as can be safely handled using 90 degree cutter blades. Then the surgeon may want to work with a short 45 degree cutter blade then one or more longer 45 degree cutter blades to remove material that would be difficult to access using a 90 degree cutter blade. Finally, in some cases, the surgeon may opt to use a short 135 degree cutter blade followed by one or more longer 135 degree cutter blades to cut nucleus material that is difficult to access using either a 90 degree or a 45 degree cutter blade.

FIG. 10 shows three views of a cutter blade 500. Visible are the cutter blade hole 407 and the cutter blade slot 427. The cutter blade arm 402 is joined to the longitudinal portions 406 by a pair of transitional sections 470. While the precise position is not particularly relevant, in the area where the two transitional sections 470 meet the two longitudinal sections 406, the two ends of the cutter blade meet. This point of contact could be deemed place where the loop is closed. However, it may be simpler to call the loop closed at 550 which is placed at cutter blade hole 407 and the currently adjacent portion of cutter blade slot 427 as those two are joined when the cutter blade is attached to the cutter assembly at the blade shaft (See FIG. 5) The closed loop adds a layer of redundancy in that in the event of a break in cutter blade 500 while inserted into an intervertebral disc space, all portions of the cutter blade 500 will remain connected to the cutter shaft through either the portion of the cutter blade with the slot 427 or the portion of the cutter blade with a hole 407. As all parts of the cutter blade are connected to the cutter shaft even after a break in the cutter blade, the parts can be removed from the intervertebral disc space by prompt removal of the cutter assembly.

Surgeons may note the break in the cutter blade either by a change in feel in the operation of the cutter or by a visible change in the cutter blade as indicated in the real-time fluoroscopic imaging.

Cutter blade 500 can be said to have six different cutting edges 504, 508, 512, 516, 520, 524. Three cutting edges 504, 508, 512 on one side and three cutting edges 516, 520, 524 on the other side. Edges 504 and 516 are on the proximal portion 536 of the cutter blade 500, that is the portion of the cutter blade 500 that is closer to the handle 416 (FIG. 4A) than the other portion of the closed loop that is the distal portion 542 of the cutter blade 500. When inserted into the intervertebral disc space, the exterior of the proximal portion 536 will generally face the endplate on the proximal vertebral body (whether or not the proximal portion is parallel to the endplate). Edges 508 and 520 are on the distal portions 542 of the cutter blade 500. When inserted into the intervertebral disc space, the exterior of the distal portion 542 will generally face the endplate on the distal vertebral body (whether or not the distal portion 542 is parallel to the endplate). Edges 512 and 524 are on the tip 548 of the cutter blade 500 between the distal portion 542 and the proximal portion 536.

Note that the sides of a cutter blade are not necessarily flat. The sides (sometimes called faces) have features that are visible when looking at that side or face of the object (just as the indentations on one of the six faces of a single die from a pair of dice are visible when looking at that face or side of the die).

In each case, the cutting edges are on the inner perimeter 552 of the closed loop rather than on the outer perimeter 556 as the outer perimeter 556 might possibly contact the cartilage on an endplate. By recessing the cutting edges relative to the outer perimeter 556 of the closed loop, the cutter blade 500 is adapted to minimize trauma to either the cartilage 356 (FIG. 8) on the proximal endplate 352 (likely to be the inferior endplate when viewed in context of the intervertebral disc space 312) or the cartilage 346 (FIG. 8) on the distal endplate 342 (likely to be the superior endplate when viewed in the context of the intervertebral disc space 312). Although the cutter blade 500 has a nominal blade angle of 90 degrees, as illustrated in FIG. 8, it would not be impossible for such a cutter blade 500 to make contact with the cartilage on the superior endplate.

By having cutting edges on both sides of cutter blade 500, the surgeon may cut nucleus material while rotating the cutter blade in the clockwise direction and also while rotating the cutter blade in the counter-clockwise direction. (Clockwise and counterclockwise are dependent on orientation. One way of defining clockwise would be as viewed from the cutter while looking from proximal towards distal end of the cutter assembly. This would match the way the surgeon would view rotation of the cutter handle.)

While being bidirectional is a useful feature, not all cutter blades must have cutting edges on both sides. Likewise as discussed below, some cutter blades may have one type of cutting edge on one side and a second type of cutter blade on the second side. While it may be advantageous for some cutter blades to have blade edges on the tips of the cutter blade (such as blade edges 512 and 524 in FIG. 10), some cutter blades may not have a blade edge in the tip or may have a different blade edge type in the tip 548 than in the distal portion 542 and proximal portion 536.

The cutting blade 500 has a gap 528 within the closed loop that may allow material to pass through the gap while the cutter blade 500 is being rotated within the intervertebral disc space 312. This may add another aspect to the cutting action while reducing the resistance to the cutter blade 500 moving through the intervertebral disc space 312. Other cutter blades may have less of a gap between the distal and proximal portions or no gap at all. A cutter blade without a gap large enough to allow material to pass through the gap in the inside perimeter of the close loop receives benefit from the closed loop as noted above in that having the closed loop connected to the cutter shaft provides two points of connection for the cutter blade and provides at least one point of connection from each part of the cutter blade to the cutter shaft 410 in the event of a break in the cutter blade.

The cutter blade 500 may be described as having a reverse bevel to place the cutting edges away from the outer perimeter. Note that while the blade edges 504, 508, 512, 516, 520, and 524 on cutter blade 500 are recessed all the way to the inner perimeter 552 of the closed loop, other cutter blades seeking to avoid damaging cartilage or endplates may recess the blade edges to be away from the outer perimeter 556 of the closed loop but not all the way to the inner perimeter 552 of the closed loop. The blade edges may, for example, be midway between the outer perimeter 556 and the inner perimeter 552 and be sufficiently recessed to avoid damaging the cartilage.

FIG. 11 shows a cross section of cutter blade 500 with blade edges 508 and 520. The bevel angle 532 may be in the range of 15 to 80 degrees, often in the range 15 to 40 degrees, often in the range of 20 to 35 degrees and sometimes 30 degrees.

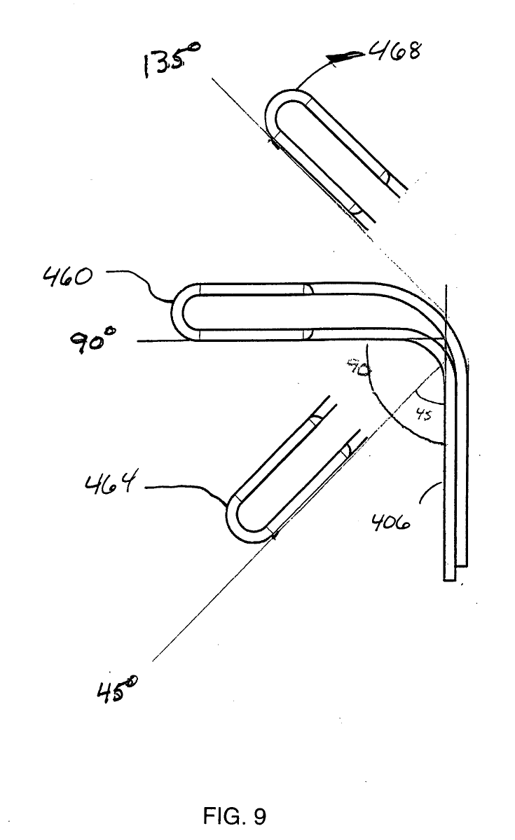

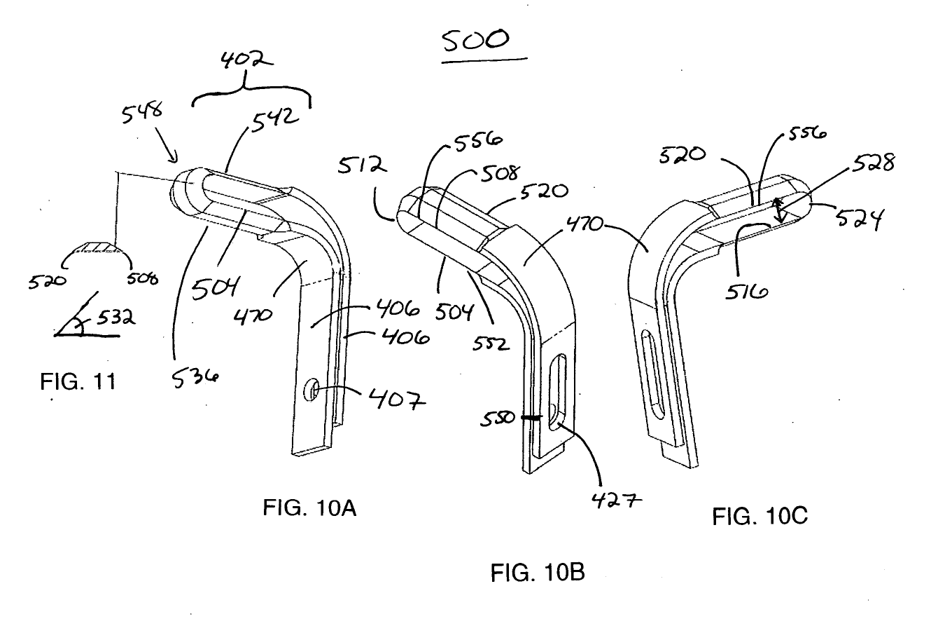

FIG. 12 and FIG. 13 illustrate two concepts of interest. Looking at cutter blade 600 in FIG. 12 and comparing it to cutter blade 500 previously discussed in FIG. 10 and shown again in FIG. 13, one difference is that the proximal blade edge 604 is not substantially parallel with distal blade edge 608 in cutter blade 600. Extensions of the two blade edges would join and form an angle of approximately 12 degrees. This is in contrast with cutter blade 500 which has proximal blade edge 504 substantially parallel to distal blade edge 508.

FIGS. 12 and 13 allow a discussion of a feature in cutter shaft 410 that was visible in FIGS. 5A and 5B. Cutter shaft 610 receives the longitudinal portion of cutter blade 600 into a slot and the cutter blade 600 may be pinned to cutter shaft 610 in the manner discussed with respect to FIGS. 5A and 5B. However, the cutter shaft 610 differs from cutter shaft 410 in that it lacks the cutter shaft extensions 480. These cutter shaft extensions 480 (sometimes called goal posts) provide additional support to the cutter blade 500. This additional support may be desired, in particular, for cutter blades with longer throws.

For some cutter blades, particularly those with shorter throws, a cutter shaft along the lines of cutter shaft 610 may be desirable in order to avoid having cutter shaft extensions 480 making contact with the cartilage 342 on the endplate 342 of the distal vertebral body 304 (See FIG. 8). This risk may be more relevant when used with a cutter blade having an angle of less than 90 degrees, for example a cutter blade with an angle of 45 degrees.

A second reason for using a cutter shaft 610 without cutter shaft extensions 480 is when using a short throw cutter blade with a desire to allow more flex in the blade. In some instances, additional flex in the shorter throw cutter blades is thought to help the cutter blade cut more effectively.

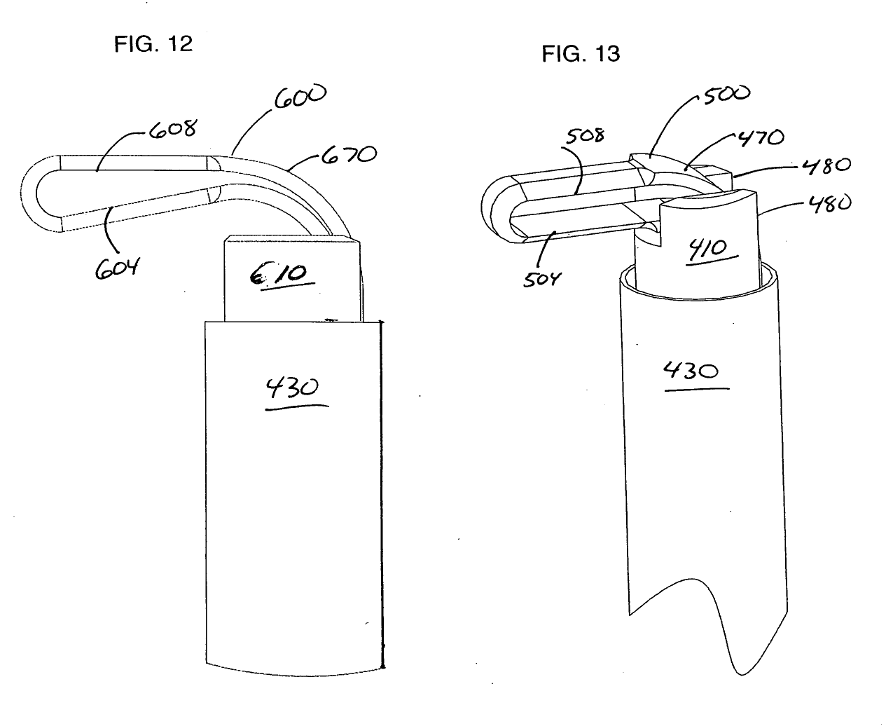

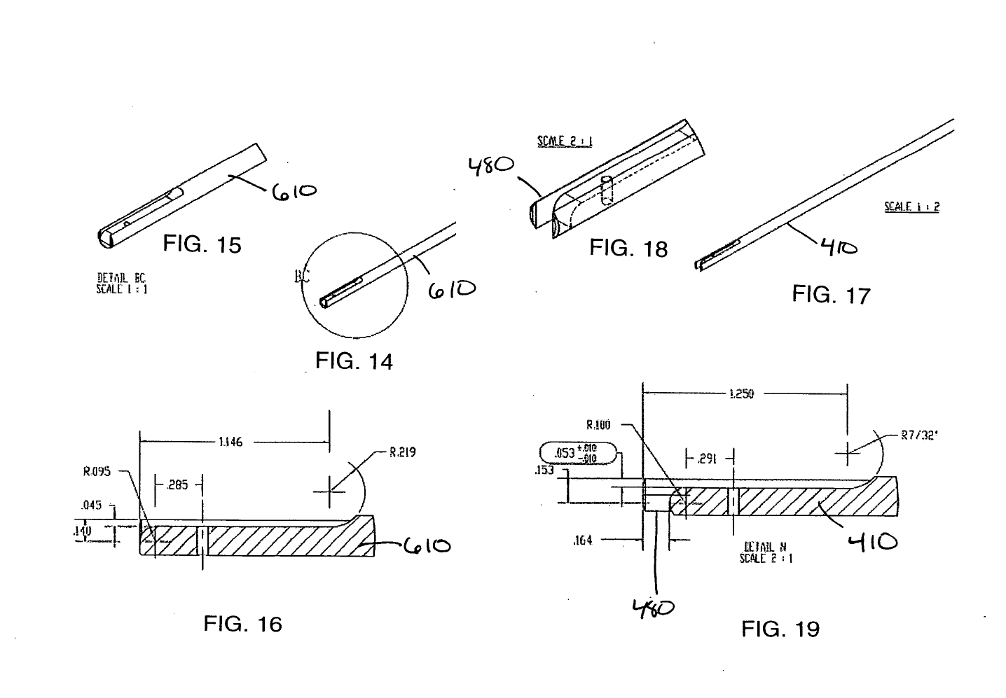

FIG. 14 is the distal end of a cutter shaft such as cutter shaft 610. FIG. 15 is an enlarged detail of FIG. 14. FIG. 16 is a cross section of the distal end of cutter shaft 610. Analogous drawings for a cutter shaft 410 with cutter shaft extensions 480 are shown in FIGS. 17-19.

Serrated Blades

While cutter blades with blade edges as shown in FIGS. 100A-10C are effective in cutting nucleus pulposus material, in some situations, another blade type may be more effective or efficient in preparing the nucleus pulposus material for removal.

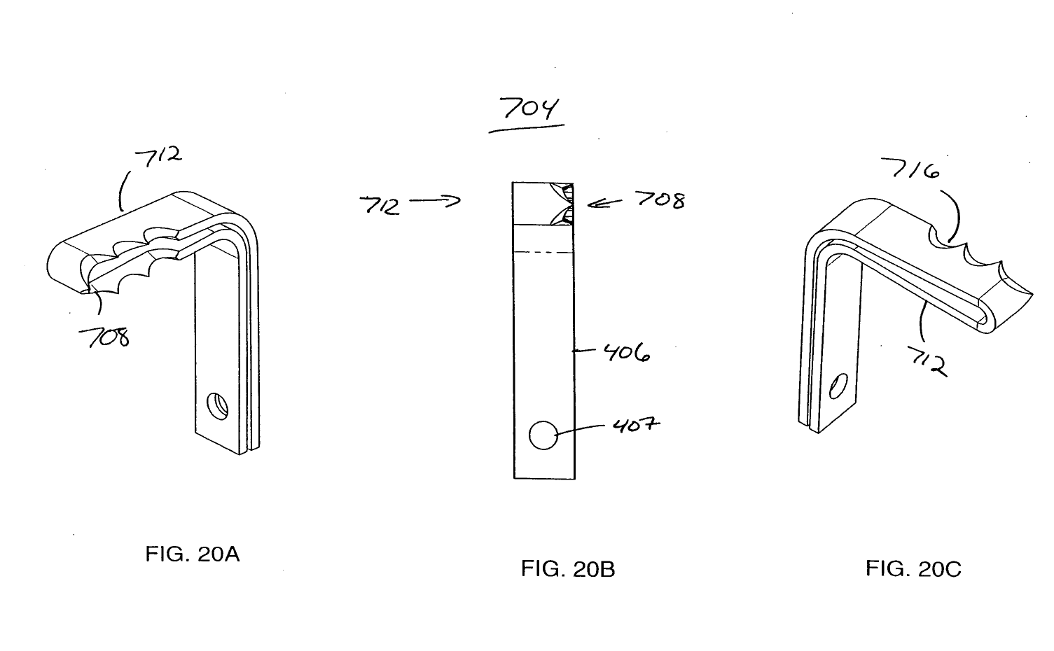

FIGS. 20A-20C show three views of a cutter blade 704 with one serrated side 708 and a flat (blunt) side 712 not intended for cutting. FIG. 20A is a top perspective view of the cutter blade viewing the serrated side 708. FIG. 20B is a front view looking at the blade arm with the longitudinal portion 406 of the cutter blade 704 and the cutter blade hole 407. FIG. 20C is a top perspective view of the flat side 712. The serration pattern uses round serrations 716.

FIGS. 21A-21C show three views of a cutter blade 720 (this drawing does not include the cutter blade hole or the cutter blade slot as the focus is on the serrated pattern). Again there is a serrated cutting side 724 and a non-cutting side 728. FIG. 21 illustrates how the serration pattern on the outside perimeter of the closed loop (serration cuts 732, 734, and 736) is offset from the serration pattern on the inside perimeter of the closed loop (serration cuts 742,744, 746). FIGS. 21A-21C highlight that often the serration pattern at the cutter blade tip 750 is different than the serration pattern elsewhere.

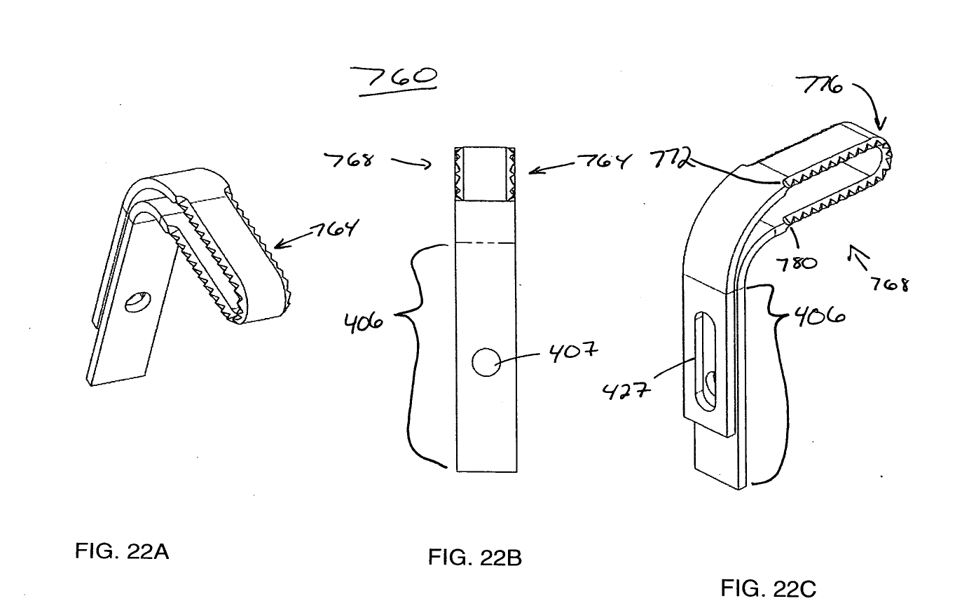

FIGS. 22A-22C show several views of a cutter blade 760 with serrated cutting edges on both the clockwise side 764 of the cutter blade 760 and counterclockwise side 768 of the cutter blade 760. FIG. 22B is a front view of the cutter blade 760 showing the cutter blade hole 407 on the longitudinal portion 406. The serration pattern is continuous from one end 772 on the distal portion of the cutter blade 760 around the blade tip 776 to the other end 780 on the proximal portion of the cutter blade 760. (Proximal and distal, respectively, in the context of the cutter handle 416 (proximal) when the cutter blade 760 (distal) is part of a cutter assembly). The serration pattern used on cutter blade 760 has a dual bevel so the tips of the serration teeth are near the midline of the thickness of the material used to make the cutter blade 760. The serrations are not round serrations as shown in FIG. 21 but a triangular serration with serration valleys between the teeth tips that come to a pronounced “V”-shape. In some instances as a cutter blade with V-shaped serrations valleys is moved relative to nucleus material, the movement of the V-shaped serration valleys relative to the nucleus material may cause localized compression of the nucleus material so that the nucleus material is effectively pinched. This effect may help the serration valleys grip nucleus material as the cutter blade continues to move and promote tearing of the nucleus material away from the cutter blade to enhance the work of the cutter blade in preparing the nucleus material for removal from the intervertebral disc space.

The net effect of the tooth pattern and dual bevels shown in the various views provided in FIG. 22A-22-C is to create a pair of cutting surfaces with essentially a series of four sided pyramids with the apexes of the pyramids aligned halfway between the outer perimeter of the closed loop and the inner perimeter of the closed loop over the cutting portion of each of the two faces of the two sided cutter blade.

One of skill in the art will appreciate that cutter blades may have the blade edges cut into flat stock before the stock is processed to assume the closed loop configuration. One of skill in the art will appreciate that the blade stock or the formed blade may need post-processing steps to remove material by polishing or an analogous process.

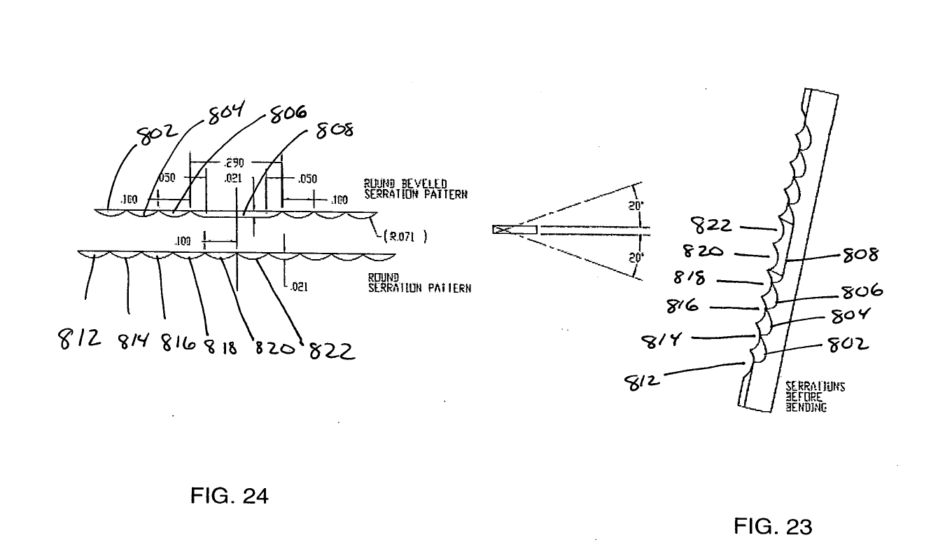

FIG. 23 and FIG. 24 show details of adding the serrations to flat stock of appropriate size. FIG. 23 is a view of the relevant portion of the sharpened stock before bending into the shape of a cutter blade.

FIG. 24 illustrates the pattern used to remove metal on each side of the blade stock to create a serration pattern such as shown in FIG. 23. As indicated to the right of the removal pattern at the top end of the blade stock 100% of the material is removed and the amount decreases with depth of cut into the blade stock. The angle for this decrease in the amount of material removed may be in the vicinity of about 20 degrees. In order to help visualize the interaction between the figures, indications for the removal of material on the first face (802, 804, 806, and 808) are shown with the indications for removal of material on the second face (812, 814, 186, 181, 820, 822). In FIG. 23, only one set of serrations is shown in the blade stock. Thus, this blade will only be able to cut when rotated in one direction (rather than being able to cut in both the clockwise and counterclockwise directions).

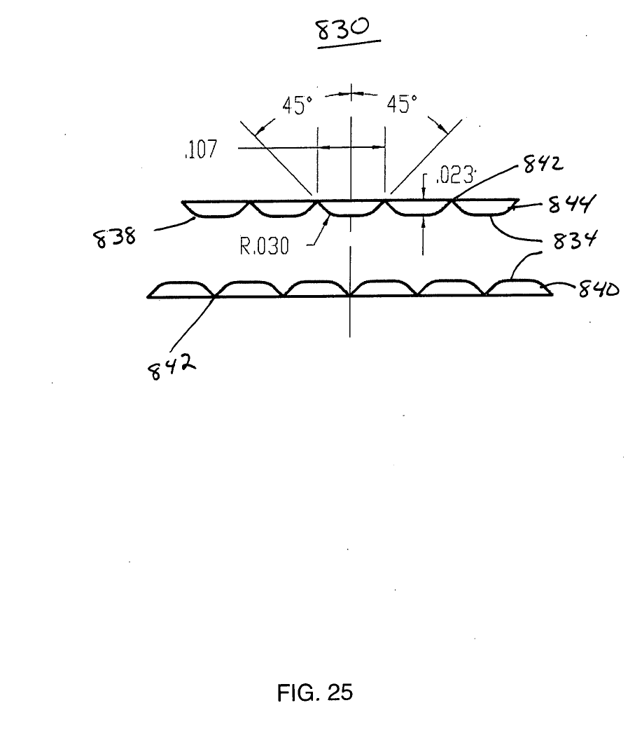

FIG. 25, like FIG. 24 notes the material to be removed to form a serration pattern for a cutter blade. FIG. 25 shows an example of a trapezoidal serration pattern 830. Each trapezoid 834 has a pair of parallel lines (top and bottom) and a pair of non-parallel lines. The corners 838 of the trapezoids cut into the blade stock may be rounded as shown here. As with the serration pattern shown in FIG. 24, the material left behind to form the tips of the serrations teeth 842 are offset so that a tooth tip on one face is aligned with the midpoint of a trapezoid on the opposing face.

In FIG. 25 and in other serrations patterns in this disclosure, one pattern of X repeats on one face is combined with X+1 repeats on the opposite face as part of the effort to stagger the teeth tips. One of skill in the art will appreciate that instead of having a pattern of five trapezoids and second pattern of six trapezoids both aligned on the same midpoint in order to achieve the desired staggered pattern that each side could have the same number of trapezoids by either removing trapezoid 840 or adding one adjacent to 844.

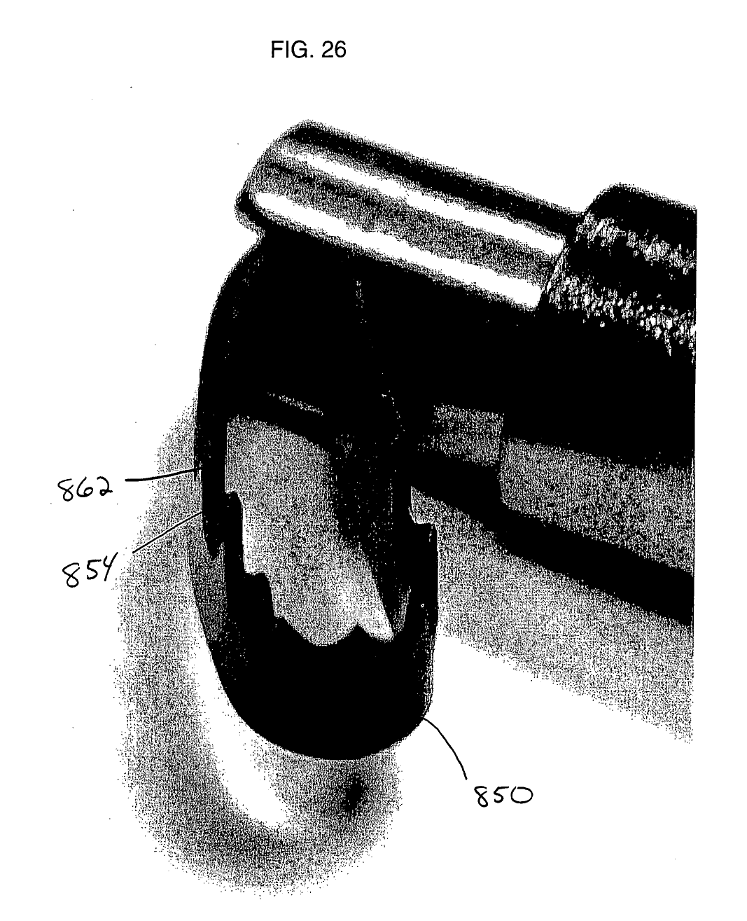

FIG. 26 shows an example of a trapezoidal serration pattern on cutter blade 850. This type of serration pattern produces a very aggressive serrated blade with serration teeth alternating between the inside perimeter of the closed loop (such as tooth tip 854) and the outside perimeter of the closed loop (such as tooth tip 862).

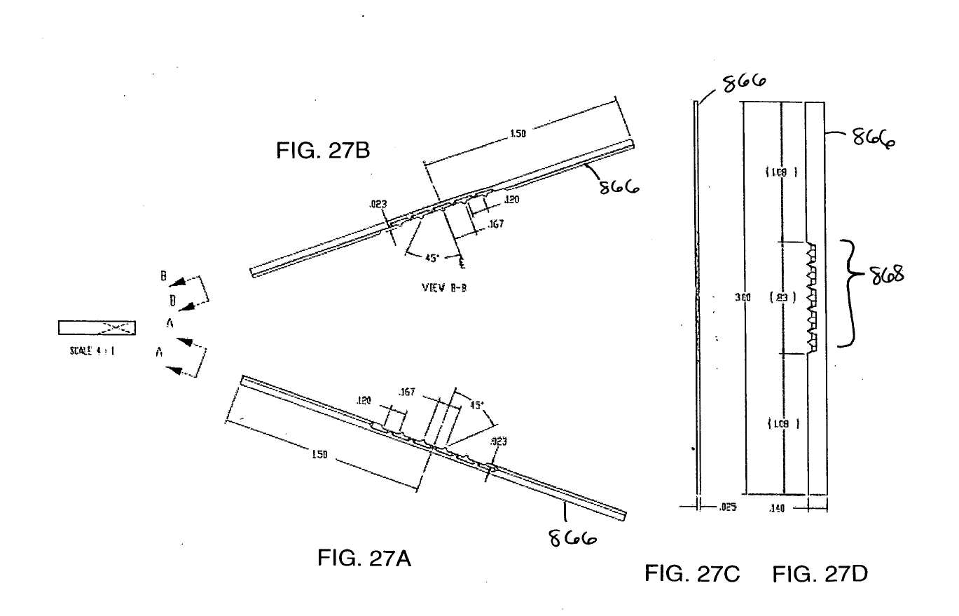

FIGS. 27A-27D show views of blade stock 866 with a trapezoidal serration pattern 868. In this pattern 868, five trapezoids are cut into one face of the blade stock 866 and six trapezoids are cut into the opposite face with the patterns offset so that a tooth on one face lines up with the midline of a trapezoid on the opposite face. One of skill in the art will appreciate that other combinations beyond 5 trapezoids and 6 trapezoids are possible and that increasing the number of trapezoids cut on each side over the fixed length to receive the serration pattern will result in a finer tooth pattern. Conversely, reducing the number of trapezoids per side over a fixed length to receive the serration pattern will receive a coarser tooth pattern. In some situations a finer tooth pattern may be preferred over a coarser tooth pattern.

As evident when viewing FIG. 26, the serration pattern may be cut across the face so that the depth of the trapezoid varies from the full depth of the blade stock down to zero. The angle for this bevel may be approximately 20 degrees but other angles could be used.

When cutting deeply into the blade stock to create an aggressive serrated pattern, it may be desirable to create a strong and durable cutter blade by either not providing a cutting surface on the opposite side (clockwise versus counter clockwise side of the cutter blade) or provide a non-serrated cutting edge such as shown in FIGS. 10A-10C. Such a hybrid cutter blade (serrated on one side and non-serrated on the other side) may be desirable as it provides two different types of cutting actions with one cutter blade (serrated/tearing action and slicing).

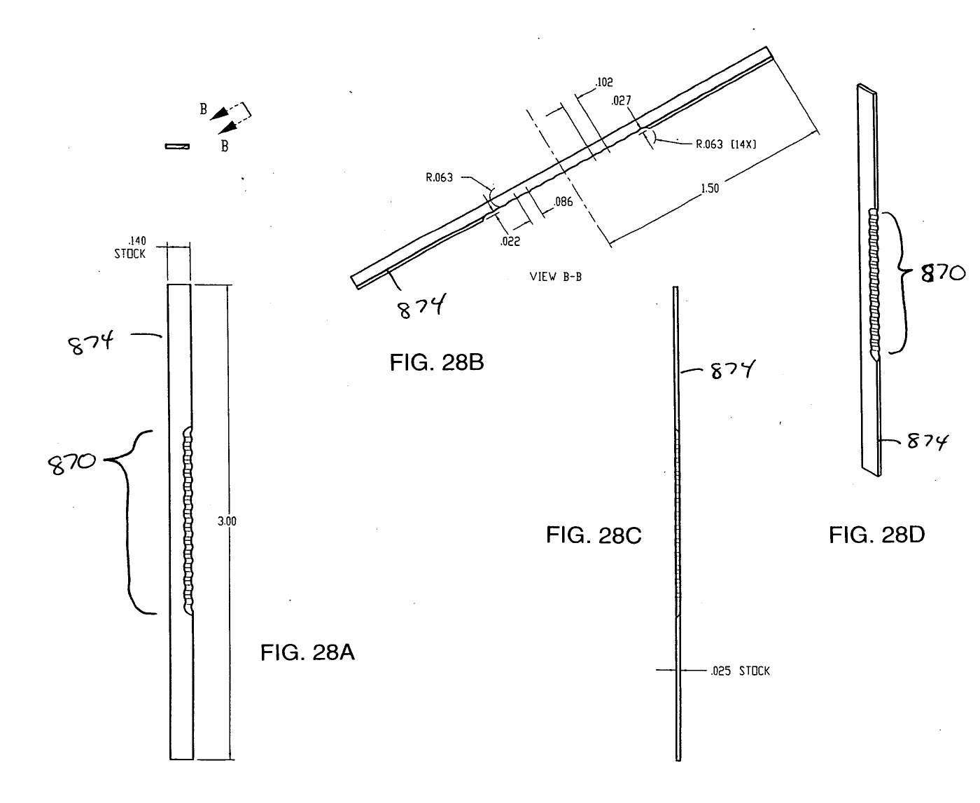

FIGS. 28A-D shows a rounded tooth serration pattern 870 cut into blade stock 874 that is 0.140 inches (3.5 mm) across, 3 inches (76 mm) long, and 0.025 inches (0.64 mm) deep. The serration pattern 870 is less severe than some of the serration patterns discussed above.

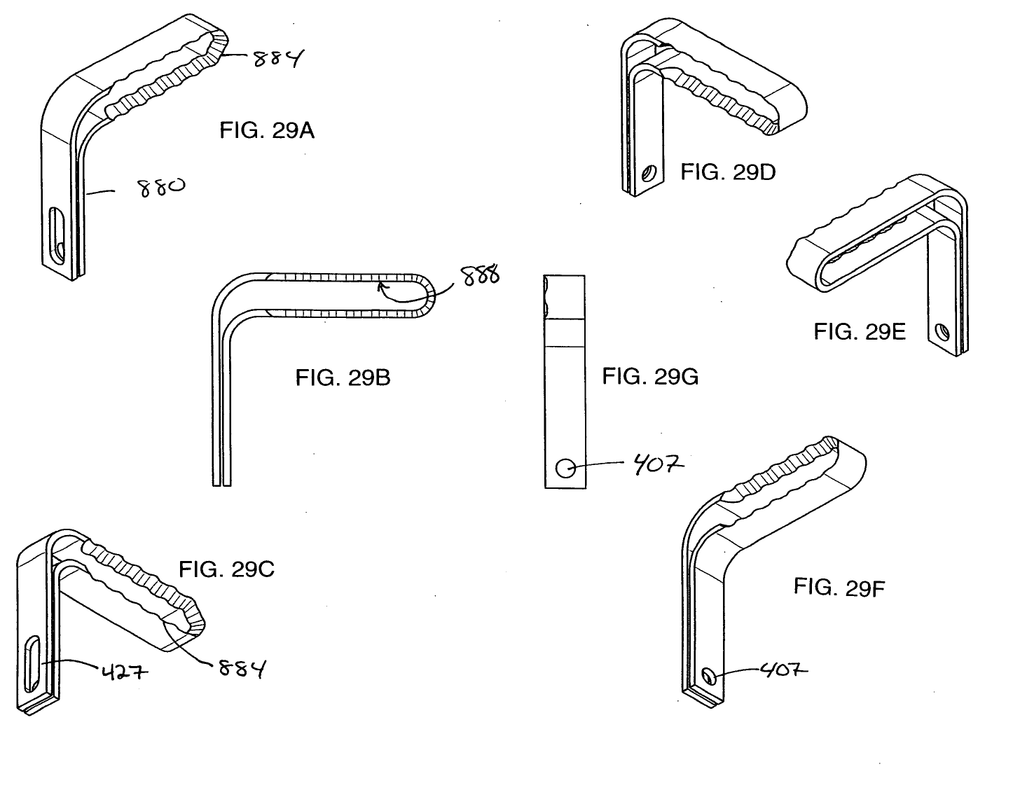

FIGS. 29A-G show a variety of views of a cutter blade 880 made from blade stock with a serrations pattern like that shown as serration pattern 870 in FIGS. 28A-D. The blade stock has been bent to position the cutting edges on the outer perimeter 884. This arrangement would tend to make cutter blade 880 more suitable to prepare an intervertebral disc space for a fusion procedure than a therapeutic procedure where bleeding of the cartilage and endplates is not desired (such as the provision of dynamic stabilization therapy, e.g., a motion preservation device). One of skill in the art will recognize that by reversing the direction of bending of the blade stock (and making the necessary corrections to the process for adding the cutter blade hole 407 and the cutter blade slot 427) that one could use this blade stock to make a cutter blade (not shown) with the cutter edge on the inside perimeter 888 of the cutter blade. Such a cutter blade may be appropriate for use in a procedure that does not want bleeding from the cartilage and endplates.

FIG. 30 shows a side view of a cutter blade 492 with a 135 degree blade angle. FIG. 31 shows a side view of a cutter blade 496 with a 134 degree blade angle where the proximal portion of the blade arm 402 is not parallel with the distal portion of the blade arm 402.

Material Choices and Other Details

In the context of the present invention, the term “biocompatible” refers to an absence of chronic inflammation response or cytotoxicity when or if physiological tissues are in contact with, or exposed to (e.g., wear debris) the materials and devices of the present invention. In addition to biocompatibility, in another aspect of the present invention it is preferred that the materials comprising the instruments are sterilizable; visible and/or imageable, e.g., fluoroscopically.

The cutter shaft and cutter sheath are typically fabricated from a metal or metal alloy, e.g., stainless steel and can be either machined or injection molded.

Due to limited disc height in certain patients, e.g., where fusion is indicated due to herniated or collapsed discs, cutter blades are preferably constructed to have a lower profile during extension, use, and retraction.

In one aspect of the present invention, the separation distance between the first and second cutting edges is a controllable variable in manufacturing (that is, predetermined during cutter blade formation, through heat treatment of the pinned, preferred nickel-titanium shape-memory alloy, e.g., Nitinol™). The separation distance between cutting edges varies from about 2 mm to about 8 mm, and, often is about 3 mm to about 4 mm. Some cutter blades have a tear drop shape. The maximum separation between cutting edges may be located within about the radially outwardly most one third of the total blade length. Alternatively, the maximum separation may be positioned within the radially inwardly most third of the blade length, or within a central region of the blade length, depending upon the desired deployment and cutting characteristics.

In accordance with one aspect of the embodiments described herein, the blade arms and the cutter blades in general can be formed from strip material that is preferably a shape memory alloy in its super-elastic or austenitic phase at room and body temperature and that ranges in width from about 0.10 inches (2.5 mm) to about 0.20 inches (5 mm) and in thickness from about 0.015 inches (0.38 mm) to about 0.050 inches (1.3 mm). Blade arms formed in accordance with the present embodiment are generally able to be flexed in excess of 100 cycles without significant shape loss, and twisted up to one and ½ full turns (about 540 degrees) without breakage. This is twisting of one end of the cutter blade relative to another portion of the cutter blade.

The shape memory feature is useful both in allowing the cutter blade to resume the extended position which is in shape memory but the shape memory helps the cutter blade resume its intended shape after being distorted while being rotated within the intervertebral disc space and receiving uneven resistance to motion.

In one embodiment, the cutting blade and cutter blade edge is formed from a super-elastic, shape memory metal alloy that preferably exhibits biocompatibility and substantial shape recovery when strained to 12%. One known suitable material that approximates the preferred biomechanical specifications for cutter blades and cutter blade edges and blade arms is an alloy of nickel and titanium (e.g., Ni₅₆—Ti₄₅ and other alloying elements, by weight), such as, for example, Nitinol strip material #SE508, available from Nitinol Devices and Components, Inc. in Fremont, Calif. This material exhibits substantially full shape recovery (i.e., recovered elongation when strained from about 6%-10%, which is substantially better than the recovered elongation at these strain levels of stainless steel).

The shape and length of the formed cutter blade in general varies for the different cutting modes. The shape memory material can be formed into the desired cutter blade configuration by means of pinning alloy material to a special forming fixture, followed by a heat-set, time-temperature process, as follows: placing the Nitinol strip (with the blade's cutting edge(s) already ground) into the forming fixture and secured with bolts; and placing the entire fixture into the oven at a temperature ranging from about 500° C. to about 550° C. (e.g., where optimum temperature for one fixture is about 525° C.) for a time ranging from between about 15 to about 40 minutes (e.g., where the optimum time for one fixture is about 20 minutes). Flexible cutter blades formed from Nitinol in this manner are particularly suited for retraction into a shaft sleeve, and are able to be extended to a right angle into the disc space. Moreover, they are able to mechanically withstand a large number of cutting “cycles” before failure would occur.

The cutting blade edges are preferably ground with accuracy and reproducibly. The angle of the inclined surface of the blade relative to the blade's flat side surface typically ranges from about 5 degrees to about 70 degrees, often about 20 degrees to about 50 degrees. In one embodiment, the blade angle is approximately 30 degrees relative to the blade's side surface.

In one aspect of the present invention, cutter blades configured with serrations are formed by a wire EDM (Electrical Discharge Machining) process to optimize design profiles. For higher manufacturing volumes, cutter blades are formed via profile grinding; progressive die stamping; machining, or conventional EDM.

In one embodiment, the shaft of the assembly is formed from solid stainless steel or other known suitable material. In one embodiment, the shaft has a diameter of approximately 0.25 inches (6.3 mm). The cutter shaft sheath may be formed from stainless steel rod or bar or other known suitable material tubing, and has a length of about 0.7 inches (17.8 mm).

As will be understood by one of skill in the art, certain components or sub-assemblies of the assemblies of the present invention may alternatively be fabricated from suitable (e.g., biocompatible; sterilizable) polymeric materials, and, for example, may be coated (e.g., with PTFE) to reduce friction, where appropriate or necessary.

For example, the cutter sheath can be fabricated from polymeric material, stainless steel, or a combination of stainless steel tubing with a low friction polymeric sleeve such as UHMWPE, HDPE, PVDF, PTFE loaded polymer. The sheath typically has an outer diameter (O.D.) of about 0.31 inches (7 mm) to about 0.35 inches (9 mm).

Alternatives

Alternative method of affixing the blade to the blade shaft.

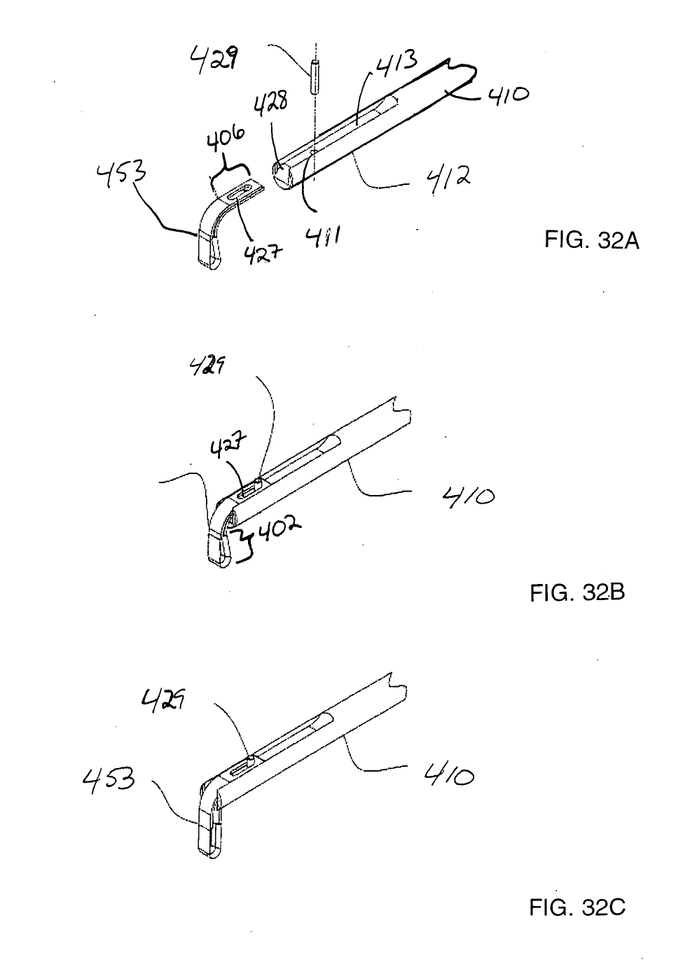

In FIGS. 32A-32B, a cutter blade 453 is placed in a shaft slot 413 in a distal end 412 of a cutter shaft 410 by a rivet 429 that passes through a cutter blade slot 427 and the cutter blade hole (407 but not visible here) and into a cutter shaft hole 411. When using a rivet, a shaft sleeve (compare element 418 in FIGS. 5A and 5B) is not required. FIG. 32C shows that this method of fixation can be combined with the goal post feature described above.

While the closed loop cutter blades disclosed above have used a cutter blade hole 407 on the longitudinal portion connected most directly to the proximal portion of the blade arm and a cutter blade slot 427 on the longitudinal portion connected most directly to the distal portion of the blade arm, one of skill in the art will appreciate that one could modify the cutter blades and the cutter shaft to allow the use of the cutter blade hole on the longitudinal portion connected most directly to the distal portion of the blade arm and the cutter blade slot on the longitudinal portion connected most directly to the proximal portion of the blade arm without deviating from the spirit of the teachings of the present disclosure.

Likewise, one could modify the cutter blades shown above to allow for at least some types of cutter blades with holes on both longitudinal portions so that once pinned there was not relative motion of one longitudinal portion relative to the other. Other non-pin attachment choices could be used that would not allow relative movement. This alternative would rely more on the ability of the shape memory material to resume a given shape as the pinned longitudinal portions could not move relative to one another to help with the transformation.

Cutter shafts may be specialized to work with specific cutter blades with specific blade angles. For example, it may be advantageous to use a cutter shaft for a 45 degree blade that allows the 45 degree blade to begin its downward angle while still in contact with the cutter shaft. Alternatively, a standard cutter shaft could be used for a range of cutter blade angles and the variation in blade angles would be handled in the cutter blades after the cutter blade has left contact with the cutter shaft. A combination of both strategies might call for a few different cutter shafts such as a 45 degree cutter shaft and a 90 degree cutter shaft and using attributes of the cutter blades to provide an expanded range of cutter blade angles.

The cutter assemblies described herein may also be used in conjunction with other methods, such as hydro-excision or laser to name just two examples to perform partial or complete nucleectomies, or to facilitate other tissue manipulation (e.g., fragmentation and/or extraction).

Alternative Handle

In accordance with one aspect of the embodiments described herein, there is provided a handle configured, for example, as a lever or pistol grip, which is affixed to the proximal end of the cutter shaft. Referring to FIG. 4B, the illustrated handle 416 is affixed to the proximal end 414 of the cutter shaft 410 by a cross-pin or set screw, which reduces the risk of handle disengagement from the cutter shaft 410 (unthreading by rotational manipulation during cutting). As mentioned, the handle 416 is preferably affixed so that it is in rotational positional alignment with the blade arm and serves as a reference marker for the blade arm's in situ orientation.

Alternatively, the handle of the cutter assembly is configured as a turn knob (not shown) fabricated from a polymeric material, such as, for example, ABS polymer or the like, that is injection moldable and that may be machined, and is affixed to the cutter shaft by means of threaded or other engagement to the cutter shaft proximal end.

Rotational Stops

In accordance with one aspect of the embodiments described herein, there are provided blade arms and cutters that are designed to be rotated and used in one direction (i.e., clockwise or counter-clockwise), i.e., the rotational motion of blade arms in only one direction (e.g., clockwise) will initiate severing of nucleus material The intended motion during the use of these blades is similar to the back and forth motion of a windshield wiper—wherein the excision with respect to these cutters occurs in the sweep that is clockwise in direction.

In one embodiment (not shown), one or more stops are placed within the cutter shaft to control blade arc or range of motion. In another embodiment (not shown), one or more stops are fitted onto the cutter sheath to control the blade arc or range of motion.

Variety of Teeth Heights

While the examples provided above have used patterns that produce teeth tips of uniform height, one of skill in the art could modify the patterns used as examples to create a pattern where some teeth are taller than other teeth.

Kits

Various combinations of the tools and devices described above may be provided in the form of kits, so that all of the tools desirable for performing a particular procedure will be available in a single package. Kits in accordance with the present invention may include preparation kits for the desired treatment zone, i.e., provided with the tools necessary for disc preparation. Disc preparation kits may differ, depending upon whether the procedure is intended to be in preparation for therapy of one or more vertebral levels or motion segments. The disc preparation kit may include a plurality of cutters. In a single level kit, anywhere from 3 to 7 cutters and, in one embodiment, 5 cutters are provided. In a two level kit, anywhere from 5 to 14 cutters may be provided, and, in one embodiment, 10 cutters are provided. The cutter assemblies will include an assortment of cutter blades. The assortment will be different depending on the specific procedure to be performed and possibly based on the patient anatomy (which may impact the range of cutter blade throw lengths needed).

Typically, a kit will include cutter assemblies with a small radial cutter blade, a medium radial cutter blade, and a large radial cutter blade. The kit will typically also include three more cutter assemblies with small, medium, and large cutter blades with a blade angle of 45 degrees. Kits for specific procedures may include other cutter assemblies with specific cutter blades for specific uses for example inclusion of cutter blades chosen for there ability to cut into and cause bleeding in either the inferior or superior endplates. All of the cutters blades are one-time use, i.e., disposable. Certain other components comprised within the cutter assembly may be disposable or reusable.

The disc preparation kit may (optionally) additionally include one or more tissue extraction tools, for removing fragments of the nucleus. In a one level kit, 3 to 8 tissue extraction tools, and, in one embodiment, 6 tissue extraction tools are provided. In a two level disc preparation kit, anywhere from about to 8 to about 14 tissue extraction tools, and, in one embodiment, 12 tissue extraction tools are provided. The tissue extraction tools may be disposable.

The cutters described above have been described in the context of use within an intervertebral disc space. One of skill in the art will recognize that the desirable attributes of the disclosed cutters could be used within other medical procedures that access material to be cut (most likely for removal before a subsequent therapeutic procedure) by delivery of a cutter blade in a sheathed state to through a lumen before the cutter blade assumes an extended position in which the cutter blade has as a shape memory. One of skill in the art will recognize that the dimensions of the cutter blade and related components may need to be adjusted to meet the relevant anatomic dimensions and the dimension of the lumen used for providing access. While there may not be cartilage covered vertebral body endplates to preserve or scrape (depending on the desired results) there may be other anatomic structures that need to be protected from cutting edges or alternatively need to be scraped as part of site preparation, thus making many of the specific teachings of the present disclosure relevant.

One of skill in the art will recognize that some of the alternative implementations set forth above are not universally mutually exclusive and that in some cases additional implementations can be created that employ aspects of two or more of the variations described above. Likewise, the present disclosure is not limited to the specific examples or particular embodiments provided to promote understanding of the various teachings of the present disclosure. Moreover, the scope of the claims which follow covers the range of variations, modifications, and substitutes for the components described herein as would be known to those of skill in the art.

What is claimed is:

1. A cutter, for disrupting material in an intervertebral space between two vertebral body endplates, the cutter adapted to extend through an axial channel with a centerline axis, the axial channel including at least one vertebral body endplate and extending into the intervertebral space,
the cutter comprising:
a cutter blade made from a shape memory material;
a first side surface on the cutter blade generally facing one of the vertebral body endplates when the cutter blade is oriented generally transverse to the centerline axis;
a second side surface on the cutter blade, separated from the first side surface by a blade thickness;
a clockwise side on the cutter blade that is the leading side when the cutter is rotated clockwise within the axial channel;
a counterclockwise side on the cutter blade that is the trailing side when the cutter is rotated clockwise within the axial channel; and
at least one cutting edge on at least one of the clockwise and counterclockwise sides.

2. A cutter blade for use in an intervertebral disc space, the cutter blade created from a shape memory material and comprising:
a first longitudinal portion of the cutter blade with a cutter blade hole for use in affixing the closed loop cutter blade to a cutter assembly;
a second longitudinal portion with a cutter blade slot for use in connecting the second longitudinal portion to the cutter assembly through a slotted connection that allows a limited range of motion of the second longitudinal portion; and
the closed loop cutter blade being retractable and extendible from a cutter sheath comprising part of the cutter assembly.

3. The closed loop cutter blade of claim 2 wherein the extended closed loop cutter blade defines a closed loop bounded when connected to the cutter assembly by a connection point running through the first longitudinal portion and the second longitudinal portion, the closed loop having an inner perimeter and an outer perimeter.

4. The closed loop cutter blade of claim 3 wherein all cutting edges are recessed from the outer perimeter.

5. The closed loop cutter blade of claim 3 wherein all cutting edges are along the inside perimeter of the closed loop.

6. The closed loop cutter blade of claim 2 wherein the closed loop cutter blade has two faces, a first face and a second face such that when the closed loop cutter blade is affixed to a cutter assembly that is rotated in a first direction, the first face is a leading face and the second face is a trailing face, and the closed loop cutter blade has a cutting edge on at least a portion of the first face and a blunt side on the second face.

7. The closed loop cutter blade of claim 2 wherein a the closed loop cutter blade has two faces, a first face and a second face such that when the closed loop cutter blade is affixed to a cutter assembly that is rotated in a first direction, the first face is a leading face and the second face is a trailing face, and the closed loop cutter blade has a cutting edge on at least a portion of the first face and has a cutting edge on at least a portion of the second face.

8. The closed loop cutter blade of claim 7 wherein the cutting edge on the first face uses a serrated cutting edge with a first pattern and the cutting edge on the second face is different from the cutting edge on the first face.

9. The closed loop cutter blade of claim 7 wherein the cutting edge on the first face uses a serrated cutting edge with a first pattern and the cutting edge on the second face uses a serrated cutting edge with a pattern different from the first pattern.

10. The closed loop cutter blade of claim 7 wherein the cutting edge on the first face uses a serrated cutting edge and the cutting edge on the second face does not.

11. The closed loop cutter blade of claim 21 wherein the closed loop cutter blade has two faces, a first face and a second face such that when the closed loop cutter blade is affixed to a cutter assembly that is rotated in a first direction, the first face is a leading face and the second face is a trailing face, and the closed loop cutter blade has a cutting edge on at least a portion of the first face and the cutting edge on the first face is formed by a pattern imposed on the a portion of the first face and a corresponding portion of the inside perimeter of the closed loop and a repetition of the pattern imposed on a portion of the first face and a corresponding portion of the outside perimeter of the closed loop.

12. The closed loop cutter blade of claim 2 wherein the closed loop cutter blade has two faces, a first face and a second face such that when the closed loop cutter blade is affixed to a cutter assembly that is rotated in a first direction, the first face is a leading face and the second face is a trailing face, and the closed loop cutter blade has a cutting edge on at least a portion of the first face and the first face has a series of four-sided pyramids with the apexes of the pyramids aligned at about halfway between the outer perimeter of the closed loop and the inner perimeter of the closed loop.

13. The closed loop cutter blade of claim 2 wherein the closed loop includes a cutting edge on a proximal portion of the cutter blade and the angle between the proximal portion of the cutter blade when in the extended position and the first longitudinal portion is between about 25 degrees and about 90 degrees.

14. The closed loop cutter blade of claim 2 wherein the closed loop includes a cutting edge on a proximal portion of the cutter blade and the angle between the proximal portion of the cutter blade when in the extended position and the first longitudinal portion is between about 90 degrees and about 155 degrees.

15. The closed loop cutter blade of claim 2 wherein the closed loop includes a cutting edge on a proximal portion of the cutter blade and the angle between the proximal portion of the cutter blade when in the extended position and the first longitudinal portion is between about 80 degrees and about 100 degrees.

16. A closed loop cutter blade for use in an intervertebral disc space, the closed loop cutter blade having shape memory of an extended position, the closed loop cutter blade having:
an inner surface which forms at least a portion of the inner perimeter of the closed loop;
an outer surface which forms at least a portion of the outer perimeter of the closed loop;
a first face on an exterior of the closed loop cutter blade between the inner surface and the outer surface, the first face being a leading face when the closed loop cutter blade is attached to a cutter assembly and rotated in a first direction around a long axis of the cutter assembly;
a second face on the exterior of the closed loop cutter blade between the inner surface and the outer surface, the second face being a trailing face when the closed loop cutter blade is attached to the cutter assembly and rotated in the first direction around the long axis of the cutter assembly; and
at least a portion of the first face having a cutting edge with a set of serrations.

17. The closed loop cutter blade of 16 wherein the first face has a first set of serrations that extend into the outer surface and a second set of serrations that extend into the inner surface.

18. The closed loop cutter blade of 17 wherein first set of serrations have serration teeth tips and the second set of serrations have serrations teeth tips that do not align with the serration teeth tips of the first set.

19. The closed loop cutter blade of 18 wherein the first set of serrations have serration pattern and the second set of serrations have the same serration pattern but offset so that the teeth tips do not align.

20. The closed loop cutter blade of 18 wherein the first set of serrations have first serration pattern and the second set of serrations have a second serration pattern different from the first serration pattern but offset so that the teeth tips do not align.

21. The closed loop cutter blade of 18 wherein the first face has valley between a serration tooth on the outer surface and a serration tooth on the inner surface.

22. The closed loop cutter blade of 21 wherein the valley contains a V formed by an acute angle.

23. The closed loop cutter blade of 16 wherein the set of serrations includes round serrations.

24. The closed loop cutter blade of 16 wherein the set of serrations includes beveled round serrations.

25. The closed loop cutter blade of 16 wherein the set of serrations includes a set of polygons.

26. The closed loop cutter blade of claim 16 wherein the set of serrations includes a set of trapezoids.

27. The closed loop cutter blade of claim 16 wherein the set of serrations is cut at an angle across the first face so that the depth of the of serrations ranges from the thickness of the first face to zero.

28. A cutter, for disrupting material in an intervertebral space between an endplate on a distal vertebral body and an endplate on a proximal vertebral body, the cutter adapted to extend through an bore along an axis extending through at least the proximal vertebral body endplate to position one end of the cutter into the intervertebral space, the cutter comprising:
a cutter shaft;
a cutter sheath surrounding at least a portion of the cutter shaft;
a cutter blade; the cutter the cutter configured to be retracted into and extended from the cutter sheath, the cutter blade having a shape memory of the extended position, the extended cutter blade significantly transverse to the long axis of the cutter shaft;
a distal side surface on the cutter blade on the side of the extended cutter blade that is adapted to be closer to the endplate on the distal vertebral body than to the endplate on the proximal vertebral body when the cutter blade is extended in the intervertebral space;
a proximal side surface on the cutter blade on the side of the inserted extended cutter blade that is adapted to be closer to the endplate on the proximal vertebral body than to the endplate on the distal vertebral body when the cutter blade is extended in the intervertebral space;
a first edge on the cutter blade that would be the leading edge of the cutter blade when the extended cutter blade is rotated in a first direction around the long axis of the cutter shaft;
a second edge on the cutter blade that would be the leading edge of the cutter blade when the extended cutter blade is rotated in a second direction around the long axis of the cutter shaft, the second direction opposite to the first direction; and
at least one cutting edge on at least one of the first or second edges, the cutting edge recessed relative to the distal side surface of the cutter blade.

29. The cutter of claim 28 wherein the cutting edge is recessed relative to the proximal side surface of the cutter blade.

30. The cutter of claim 28 wherein the cutter blade includes a loop extending from a portion of the cutter blade connected to the cutter shaft, such that a first portion of the loop contains the distal side surface and a second portion of the loop contains the proximal side surface, the loop also including a loop tip connecting the first portion and the second portion, the loop defining an open area such that when the cutter blade is rotated around the long axis of the cutter shaft.

31. The cutter of claim 28 wherein the cutter blade includes a loop extending from a portion of the cutter blade connected to the cutter shaft, such that a first portion of the loop contains the distal side surface and a second portion of the loop contains the proximal side surface, the loop also including a loop tip connecting the first portion and the second portion, the loop defining an open area such that when the cutter blade is rotated around the long axis of the cutter shaft, material may pass through the open area.

32. The cutter of claim 28 wherein the extended cutter blade significantly transverse to the long axis of the cutter shaft is positioned to be between about 25 degrees to about 155 degrees off of the long axis of the cutter shaft.

33. The cutter of claim 28 where the distal portion of the cutter blade is substantially parallel to the proximal portion of the cutter blade.

34. The cutter of claim 28 where a projection of the distal portion of the cutter blade intersects a projection of the proximal portion of the cutter blade to form an acute angle.

35. The cutter of claim 28 wherein a cutting edge on the first edge of the cutter blade is serrated.

36. The cutter of claim 35 wherein cutter blade has a series of teeth and the teeth are substantially the same height.

37. The cutter of claim 35 wherein cutter blade has a series of teeth, with a set of teeth at a first tooth height, and a set of teeth at a second tooth height, the second tooth height being greater than the first tooth height.

38. The cutter of claim 35 where the cutter blade has a serration pattern with valleys in the serration pattern having acute angles.

39. The cutter of claim 35 wherein the distal side surface and the proximal side surface are joined by a loop tip so that the cutter with a cutter blade in an extended position forms a closed loop and a cutting edge on a first edge of the cutter blade has a serration pattern along the loop tip that is different from the serration pattern found on another part of the first edge.

40. The cutter of claim 35 wherein a cutting edge on the second edge of the cutter blade is not serrated.

41. A cutter, for disrupting material in an intervertebral space between an endplate on a distal vertebral body and an endplate on a proximal vertebral body, the cutter adapted to extend through an axial bore along an axis extending through at least the proximal vertebral body endplate to position one end of the cutter into the intervertebral space, the cutter comprising:
a cutter shaft having a long axis;
a cutter sheath surrounding at least a portion of the cutter shaft;
a cutter blade; the cutter the cutter configured to be retracted into and extended from the cutter sheath the cutter blade extended generally transverse to the long axis of the cutter shaft;
a distal side surface on the cutter blade on the side of the extended cutter blade that is adapted to be closer to the endplate on the distal vertebral body than to the endplate on the proximal vertebral body when the cutter blade is extended in the intervertebral space;
a proximal side surface on the cutter blade on the side of the inserted extended cutter blade that is adapted to be closer to the endplate on the proximal vertebral body than to the endplate on the distal vertebral body when the cutter blade is extended in the intervertebral space;
the distal side surface and the proximal side surface are joined by a loop tip so that the cutter with a cutter blade in an extended position forms a closed loop; and
the cutter shaft having extensions so that a portion of an extended cutter blade that is generally transverse the long axis of the cutter shaft is located between the cutter shaft extensions.

42. A kit for preparing an intervertebral disc space for a therapeutic procedure, the kit comprising:
a cutter assembly with radial cutter blade of a first throw length;
a cutter assembly with a cutter blade having a blade angle of less than 90 degrees and the first throw length;
a cutter assembly with a radial cutter blade of a second throw length, longer than the first throw length; and
a cutter assembly with a cutter blade having a blade angle of less than 90 degrees and the second throw length.

43. The kit of claim 42 including:
a cutter assembly with a radial cutter blade of a third throw length, longer than the second throw length; and
a cutter assembly with a cutter blade having a blade angle of less than 90 degrees and the third throw length.

44. The kit of 42 wherein the blade angle for the cutter assembly with a cutter blade having a blade angle of less than 90 degrees and the first throw length is about 45 degrees.
